(19) United States
(12) Patent Application Publication
(10) Pub. No.: US 2004/0267028 A1
(43) Pub. Date: Dec. 30, 2004

(54) Preparation and use of pyrrole derivatives for treating obesity

(76) Inventors: Roger A Smith, Madison, CT (US); Harold C. Kluender, Trumbull, CT (US); Ning Su, Hamden, CT (US); Rico C. Lavoie, Hamden, CT (US); Jianmei Fan, Hamden, CT (US)

Correspondence Address: JEFFREY M. GREENMAN, BAYER PHARMACEUTICALS CORPORATION, 400 MORGAN LANE, WEST HAVEN, CT, 06516, US

(21) Appl. No.: 10/489,031

(22) PCT Filed: Sep. 24, 2002
(86) PCT No.: PCT/US02/30543

Related U.S. Application Data
(60) Provisional application No. 60/324,441, filed on Sep. 24, 2001.

(51) Int. Cl. C 07D 2 7/335

(52) U.S. Cl. 548/537

(57) ABSTRACT

This invention relates to pyrrole derivatives which have been found to suppress appetite and induce weight loss. The invention also provides methods for synthesis of the compounds, pharmaceutical compositions comprising the compounds, and methods of using such compositions for inducing weight loss and treating obesity and obesity-related disorders.

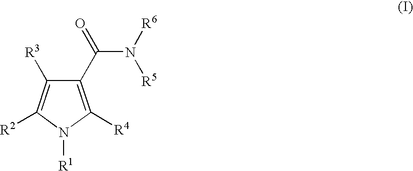

This application claims benefit of U.S. Provisional Application Ser. No. 60/324,441, filed Sep. 24, 2001, the contents of which are incorporated herein by reference in their entirety.

### FIELD OF THE INVENTION

This invention relates to the field of pharmaceuticals, in particular to the field of obesity treatment. More specifically, it relates to certain pyrrole compounds which are useful in the treatment of obesity and obesity-related disorders, and as weight-loss and weight-control agents.

### BACKGROUND OF THE INVENTION

Obesity, which is defined as an excess of body fat relative to lean body mass, is a well-established risk factor for a number of potentially life-threatening diseases such as atherosclerosis, hypertension, diabetes, stroke, pulmonary embolism, sleep apnea, and cancer. Furthermore, it complicates numerous chronic conditions such as respiratory diseases, osteoarthritis, osteoporosis, gall bladder disease, and dyslipidemias. The enormity of this problem is best reflected in the fact that death rates escalate with increasing body weight. More than 50% of all-cause mortality is attributable to obesity-related conditions once the body mass index (BMI) exceeds 30 kg/m², as seen in 35 million Americans (Lee, JAMA 268:2045-2049, 1992). By contributing to greater than 300,000 deaths per year, obesity ranks second only to tobacco smoking as the most common cause of potentially preventable death (McGinnis, JAMA 270:2207-2212, 1993). Accompanying the devastating medical consequences of this problem is the severe financial burden placed on the health care system in the United States. It is estimated that 30-50% of the middle-age population may be considered as obese (Kuczmarski et al., JAMA 272:205-211, 1994). The economic impact of obesity and its associated illnesses from medical expenses and loss of income are reported to be in excess of $68 billion/a year (Colditz, Am. J. Clin. Nutr. 55:503S-507S, 1992). This figure does not include the greater than $30 billion per year spent on weight loss foods, products, and programs (Wolf, Pharmacoeconomics. 5:34-37, 1994).

The accumulation or maintenance of body fat bears a direct relationship to caloric intake. Comprehensive treatment programs, therefore, focused on behavior modifications to reduce caloric intake and increase physical activity using a myriad of systems. These methods have limited efficacy and are associated with recidivism rates exceeding 95% (NIH Technology Assessment Conference Panel, Ann. Intern. Med. 119:764-770, 1993).

Obesity has also been treated by administering specific agents, for example, anorectic agents, to obese subjects. However, anorectic agents such as dextroamphetamine, the combination of the non-amphetamine drugs phentermine and fenfluramine (Phen-Fen), and dexfenfluramine (Redux) alone, are associated with serious side effects. Indigestible materials such as olestra (OLEAN®, mineral oil or neopentyl esters (see U.S. Pat. No. 2,962,419)) have been proposed as substitutes for dietary fat. Garcinia acid and derivatives thereof have been described as treating obesity by interfering with fatty acid synthesis. Swellable crosslinked vinyl pyridine resins have been described as appetite suppressants via the mechanism of providing non-nutritive bulk (see, e.g., U.S. Pat. No. 2,923,662).

Surgical interventions, such as gastric partitioning procedures, jejunoileal bypass, and vagotomy, have also been developed to treat severe obesity (Greenway, Endo. Metab. Clin. N. Amer. 25:1005-1027, 1996). Although these surgical procedures are somewhat more effective in the long run, the acute risk benefit ratio has reserved these invasive procedures for morbidly obese patients according to the National Health Institutes (NIH) consensus conference on obesity surgery (BMI>40 kg/m²) (NIH Conference, Ann. Intern. Med. 115:956-961, 1991). Therefore, this approach is not an alternative for the majority of overweight patients unless and until they become profoundly obese and are suffering the attendant complications.

Thus, new methods and compositions that promote weight-loss are urgently needed.

### SUMMARY OF THE INVENTION

The present invention provides substituted pyrrole derivatives which have been found to suppress appetite in laboratory animals. The invention also provides methods for synthesis of the compounds, pharmaceutical compositions comprising the compounds, and methods of using such compositions for suppressing appetite, inducing weight loss and treating obesity and obesity-related disorders.

### DETAILED DESCRIPTION OF THE INVENTION

The present invention relates to substituted pyrrole derivatives that have utility in the treatment of obesity. The invention relates to the compound of Formula (I)

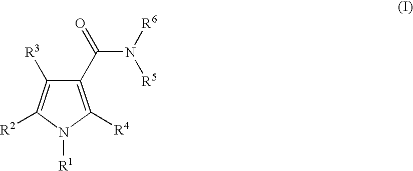

wherein

R¹ and R² are each a phenyl group, optionally substituted with one or more halogen, (C₁-C₆)alkyl, (C₁-C₆)alkoxy, trifluoromethyl, hydroxy, cyano, or nitro;

R³ is hydrogen;

R⁴ is CH₃;

R⁵ is hydrogen or (C₁-C₆)alkyl;

R⁶ is cyclohexyl which is substituted with one or more (C₁-C₃)alkyl, hydroxy, benzyloxy, (C₁-C₆)alkoxy, (C₁-C₆)alkyl-amino, bis[(C₁-C₃)alkyl]-amino, or fluorine,

(C₁-C₅)alkyl, optionally substituted with one or more cyclo(C₃-C₇)alkyl, hydroxy, benzyloxy, (C₁-C₆)alkoxy, (C₁-C₆)alkyl-amino, bis[(C₁-C₃)alkyl]-amino, or fluorine,

cyclopentyl, cycloheptyl or cyclo(C₃-C₇)alkyl-(C₁-C₃)alkyl, each of which may be optionally substituted with one or more (C₁-C₃)alkyl, hydroxy, benzyloxy, (C₁-C₆)alkoxy, (C₁-C₆)alkyl-amino, bis[(C₁-C₃)alkyl]-amino, or fluorine,

benzyl which is substituted on the phenyl ring with one or more fluorine, bromine, (C₁-C₆)alkyl, (C₁-C₆)alkoxy, trifluoromethyl, cyano, hydroxy, benzyloxy, or nitro,

phenyl substituted with one or more (C₁-C₆)alkyl, (C₁-C₆)alkoxy, trifluoromethyl, cyano, hydroxy, benzyloxy, nitro, or halogen,

piperidin-4-yl, piperidin-3-yl, or pyrrolidin-3-yl, each of which may be optionally substituted on the nitrogen atom of the piperidine or pyrrolidine ring with (C₁-C₆)alkyl, hydroxy-substituted (C₁-C₆)alkyl, or a benzyl or phenyl group that is optionally substituted on the phenyl ring with one or more of (C₁-C₆)alkyl, (C₁-C₆)alkoxy, trifluoromethyl, cyano, hydroxy, or halogen,

—NR⁷R⁸

where R⁷ is hydrogen or (C₁-C₆)alkyl;

R⁸ is (C₁-C₉)alkyl, or a phenyl group that is optionally substituted with one or more (C₁-C₆)alkyl, (C₁-C₆)alkoxy, hydroxy-substituted (C₁-C₆)alkyl, hydroxy, trifluoromethyl, cyano, nitro, or halogen; or

R⁷ and R⁸, taken together with the nitrogen atom to which they are attached, form a 5- to 10-membered saturated heterocyclic radical which is optionally substituted by one or more (C₁-C₆)alkyl, (C₁-C₆)alkoxy, hydroxy-substituted (C₁-C₃)alkyl, benzyl, phenyl, hydroxy, or fluorine; or

R⁵ and R⁶, taken together with the nitrogen atom to which they are attached, form a 5- to 10-membered saturated heterocyclic radical containing at least one additional nitrogen atom, wherein

one or more of the carbon atoms of the heterocyclic radical is optionally substituted with (C₁-C₆)alkyl, (C₁-C₆)alkoxy, hydroxy, trifluoromethyl, or fluorine, and wherein

one or both of the additional nitrogen atoms of the heterocyclic radical is optionally substituted with (C₂-C₆)alkyl, and wherein

any carbon or nitrogen atom of the heterocyclic radical is optionally substituted with 2-pyridinyl, 3-pyridinyl, 4-pyridinyl, or a benzyl or phenyl group that is optionally substituted on the phenyl ring with one or more (C₁-C₆)alkyl, hydroxy, (C₁-C₆)alkoxy, trifluoromethyl, cyano, nitro, or halogen; or

R⁵ and R⁶, taken together with the nitrogen atom to which they are attached, form a 1-piperidinyl, 1-pyrrolidinyl, or 1-morpholino group, which is substituted with one or more (C₁-C₆)alkyl, (C₁-C₆)alkoxy, hydroxy, trifluoromethyl, fluorine, or a benzyl or phenyl group that is optionally substituted on the phenyl ring with one or more (C₁-C₆)alkyl, hydroxy, (C₁-C₆)alkoxy, trifluoromethyl, cyano, nitro, or halogen.

Another embodiment of this invention are substituted pyrrole derivatives that have utility in the treatment of obesity, said derivatives having Formula I

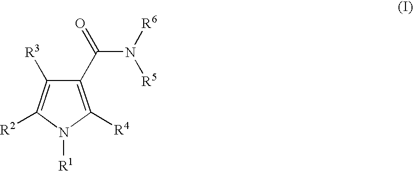

wherein

R¹ and R² are each a phenyl group optionally substituted with one or more halogen, (C₁-C₆)alkyl, (C₁-C₆)alkoxy, trifluoromethyl, cyano, or nitro;

R³ is hydrogen, (C₁-C₆)alkyl, or benzyl; and R⁴ is (C₂-C₆)alkyl or NH₂; or

R³ is (C₁-C₆)alkyl or benzyl; and R⁴ is CH₃;

R⁵ is hydrogen or (C₁-C₆)alkyl;

R⁶ is (C₁-C₉)alkyl, which is optionally substituted with one or more hydroxy, benzyloxy, (C₁-C₆)alkoxy, (C₁-C₆)alkyl-amino, bis[(C₁-C₃)alkyl]-amino, or fluorine,

benzyl, which is optionally substituted on the phenyl ring with one or more halogen, (C₁-C₆)alkyl, (C₁-C₆)alkoxy, trifluoromethyl, cyano, hydroxy, benzyloxy, or nitro,

phenyl substituted with one or more (C₁-C₆)alkyl, (C₁-C₆)alkoxy, trifluoromethyl, cyano, hydroxy, benzyloxy, nitro, or halogen,

piperidin-4-yl, piperidin-3-yl, or pyrrolidin-3-yl, each of which may be optionally substituted on the nitrogen atom of the piperidine or pyrrolidine ring with (C₁-C₆)alkyl, hydroxy-substituted (C₁-C₆)alkyl, or a benzyl or phenyl group that is optionally substituted on the phenyl ring with one or more (C₁-C₆)alkyl, (C₁-C₆)alkoxy, trifluoromethyl, cyano, hydroxy, or halogen,

—NR⁷R⁸

where R⁷ is hydrogen or (C₁-C₆)alkyl;

R⁸ is (C₁-C₉)alkyl, or a phenyl group that is optionally substituted with one or more (C₁-C₆)alkyl, (C₁-C₆)alkoxy, hydroxy-substituted (C₁-C₆)alkyl, hydroxy, trifluoromethyl, cyano, nitro, or halogen; or

R⁷ and R⁸, taken together with the nitrogen atom to which they are attached, form a 5- to 10-membered saturated heterocyclic radical which is optionally substituted by one or more (C₁-C₆)alkyl, (C₁-C₆)alkoxy, hydroxy-substituted (C₁-C₃)alkyl, benzyl, phenyl, hydroxy, or fluorine; or

R⁵ and R⁶, taken together with the nitrogen atom to which they are attached, form a 5- to 10-membered saturated heterocyclic radical, optionally substituted with one or more (C₁-C₆)alkyl, (C₁-C₆)alkoxy, hydroxy, trifluoromethyl, fluorine, or a benzyl or phenyl group that is optionally substituted on the phenyl ring with one or more (C₁-C₆)alkyl, hydroxy, (C₁-C₆)alkoxy, trifluoromethyl, cyano, nitro, or halogen.

The terms identified above have the following meaning throughout:

“Halogen” means fluorine, chlorine, bromine, or iodine.

The terms “(C₁-C₃)alkyl,” “(C₁-C₅)alkyl,” “(C₁-C₆)alkyl,”(C₁-C₉)alkyl,” and “(C₂-C₆)alkyl” mean C₁-C₃, C₁-C₅, C₁-C₆, C₁-C₉, and C₂-C₆ linear or branched alkyl groups, respectively, that may also include a cyclic alkyl radical as part of the alkyl group. For example, this includes groups such as cyclopropyl, cyclohexyl, cyclopropyl-methyl, and cycloheptyl-methyl groups. The preferred alkyl groups are methyl, ethyl, propyl, and isopropyl groups.

The term “cyclo(C₃-C₇)alkyl” means a cyclic (C₃-C₇)alkyl group, such as, for example, cyclopropyl, cyclopentyl, or cyclohexyl.

The term “(C₁-C₆)alkoxy” means a (C₁-C₆)alkyl-oxy group.

The term “5- to 10-membered saturated heterocyclic radical” means a fused or bridged, mono-, bi-, or tricyclic, non-aromatic heterocyclic radical which may contain one to three of the heteroatoms nitrogen, oxygen, or sulfur. These radicals include the following radicals, for example, pyrrolidin-1-yl, piperidin-1-yl, piperazin-1-yl, hexahydroazepin-1-yl, azepanyl-1, morpholin-4-yl, and thiomorpholin-4-yl.

When any moiety is described as being substituted, it can have one or more of the indicated substituents that can be located at any available position on the moiety. When there are two or more substituents on any moiety, each term shall be defined independently of any other in each occurrence.

Representative salts of the compounds of Formula I include the conventional non-toxic salts and the quaternary ammonium salts which are formed, for example, from inorganic or organic acids or bases by means well known in the art. For example, such acid addition salts include acetate, adipate, alginate, ascorbate, aspartate, benzoate, benzenesulfonate, bisulfate, butyrate, citrate, camphorate, camphorsulfonate, cinnamate, cyclopentanepropionate, digluconate, dodecylsulfate, ethanesulfonate, fumarate, glucoheptanoate, glycerophosphate, hemisulfate, heptanoate, hexanoate, hydrochloride, hydrobromide, hydroiodide, 2-hydroxyethanesulfonate, itaconate, lactate, maleate, mandelate, methanesulfonate, 2-naphthalenesulfonate, nicotinate, nitrate, oxalate, pamoate, pectinate, persulfate, 3-phenylpropionate, picrate, pivalate, propionate, succinate, sulfonate, tartrate, thiocyanate, tosylate, and undecanoate.

Base salts include alkali metal salts such as potassium and sodium salts, alkaline earth metal salts such as calcium and magnesium salts, and ammonium salts with organic bases such as dicyclohexylamine salts and N-methyl-D-glucamine. Additionally, basic nitrogen containing groups may be quaternized with such agents as lower alkyl halides such as methyl, ethyl, propyl, and butyl chlorides, bromides and iodides; dialkyl sulfates like dimethyl, diethyl, and dibutyl sulfate; and diamyl sulfates, long chain halides such as decyl, lauryl, myristyl and strearyl chlorides, bromides and iodides, aralkyl halides like benzyl and phenethyl bromides and others.

The esters in the present invention are non-toxic, pharmaceutically acceptable ester derivatives of the alcohols of Formula I. This includes ester derivatives prepared from acetic, benzoic, mandelic, stearic, lactic, salicylic, hydroxynaphthoic, glucoheptonic, and gluconic acid. The alcohol compounds of Formula I may be esterified by a variety of conventional procedures including reacting the appropriate anhydride, carboxylic acid, or acid chloride with the alcohol group of the Formula I compound. The appropriate anhydride is reacted with the alcohol in the presence of an acylation catalyst such as 1,8-bis[dimethylamino]naphthalene or DMAP (N,N-dimethylaminopyridine). An appropriate carboxylic acid may be reacted with the alcohol in the presence of a dehydrating agent such as dicyclohexylcarbodiimide, 1-[3-dimethylaminopropyl]-3-ethylcarbodiimide or other water soluble dehydrating agents which are used to drive the reaction by the removal of water, and optionally, an acylation catalyst. Esterification may also be reached using the appropriate carboxylic acid in the presence of trifluoroacetic anhydride and optionally, pyridine, or in the presence of N,N-carbonyldiimidazole with pyridine. Reaction of an acid chloride with the alcohol may be carried out with an acylation catalyst such as DMAP or pyridine. One skilled in the art would readily know how to successfully carry out these as well as other methods of esterification of alcohols. Sensitive or reactive groups on the compound of Formula I may need to be protected during any of the above methods for forming esters, and protecting groups may be added and removed by conventional methods well known in the art.

It will be appreciated that diastereomers and enantiomers of the exemplified structures will often be possible, and that pure isomers represent preferred embodiments. It is intended that pure stereoisomers, and mixtures thereof, are within the scope of the invention.

The compounds of this invention may, either by nature of asymmetric centers or by restricted rotation, be present in the form of isomers. Any isomer may be present in the (R)-, (S)-, or (R,S) configuration, preferably in the (R)- or (S)- configuration, whichever is most active.

All isomers, whether separated, pure, partially pure, or in racemic mixture, of the compounds of this invention are encompassed within the scope of this invention. The purification of said isomers and the separation of said isomeric mixtures may be accomplished by standard techniques known in the art.

Geometric isomers by nature of substituents about a double bond or a ring may be present in cis (=Z-) or trans (=E-) form, and both isomeric forms are encompassed within the scope of this invention.

The particular process to be utilized in the preparation of the compounds of this invention depends upon the specific compound desired. Such factors as the selection of the specific moieties and the specific substituents on the various moieties, all play a role in the path to be followed in the preparation of the specific compounds of this invention. These factors are readily recognized by one of ordinary skill in the art.

For synthesis of any particular compound, one skilled in the art will recognize that the use of protecting groups may be required for the synthesis of compounds containing certain substituents. A description of suitable protecting groups and appropriate methods of adding and removing such groups may be found in: Protective Groups in Organic Synthesis, Second Edition, T. W. Greene, John Wiley and Sons, New York, 1991.

In the Reaction Schemes below, one skilled in the art will recognize that reagents and solvents actually used may be selected from several reagents and solvents well known in the art to be effective equivalents. When specific reagents or solvents are shown in a Reaction Scheme, therefore, they are meant to be illustrative examples of conditions desirable for the execution of that particular Reaction Scheme. Abbreviations not identified in accompanying text are listed later in this disclosure under “Abbreviations and Acronyms.”

Another object of this invention is to provide methods of making the compounds of the invention. The compounds may be prepared from readily available materials by the methods outlined in Reaction Schemes 1 and 2 below, and by obvious modifications thereto.

The present invention relates to the use of the compounds of this invention for the treatment of bulimia and obesity including associated dyslipidemia and other obesity- and overweight-related complications such as, for example, cholesterol gallstones, cancer (e.g., colon, rectum, prostate, breast, ovary, endometrium, cervix, gallbladder, and bile duct), menstrual abnormalities, infertility, polycystic ovaries, osteoarthritis, and sleep apnea, as well as for a number of other pharmaceutical uses associated therewith, such as the regulation of appetite and food intake, dyslipidemia, hypertriglyceridemia, Syndrome X type II diabetes (non-insulin-dependent diabetes), atherosclerotic diseases such as heart failure, hyperlipidemia, hypercholesteremia, low HDL levels, hypertension, cardiovascular disease (including atherosclerosis, coronary heart disease, coronary artery disease, and hypertension), cerebrovascular disease and peripheral vessel disease. The compounds of this invention may also be useful for treating physiological disorders related to, for example, regulation of insulin sensitivity, inflammatory response, plasma triglycerides, HDL, LDL, and cholesterol levels and the like.

The compounds of Formula I of this invention are expected to be valuable as therapeutic agents. Accordingly, an embodiment of this invention includes a method of treating the various conditions identified above in a patient (including mammals) which comprises administering to said patient a composition containing an amount of the compound of Formula I that is effective in treating the target condition.

Compounds of Formula I may be administered alone or in combination with one or more additional therapeutic agents. Combination therapy includes administration of a single pharmaceutical dosage formulation which contains a compound of Formula I and one or more additional therapeutic agents, as well as administration of the compound of Formula I and each additional therapeutic agents in its own separate pharmaceutical dosage formulation. For example, a compound of Formula I and a therapeutic agent may be administered to the patient together in a single oral dosage composition such as a tablet or capsule, or each agent may be administered in separate oral dosage formulations.

Where separate dosage formulations are used, the compound of Formula I and one or more additional therapeutic agents may be administered at essentially the same time (e.g., concurrently) or at separately staggered times (e.g., sequentially).

For example, the compounds of Formula I may be used in combination with other therapies and drugs useful for the treatment of obesity, for example, in combination with β₃-adrenoreceptor agonists such as CL-316,243, or in combination with a drug compound that modulates digestion and/or metabolism such as drugs that modulate thermogenesis, lipolysis, gut motility, fat absorption, and satiety.

In addition, the compounds of Formula I may be administered in combination with one or more of the following hypoglycemic agents for the treatment of diabetes or diabetes-related disorders: insulin; biguanidines such as metformin or buformin; sulfonylureas such as acetohexamide, chloropropamide, tolazamide, tolbutamide, glyburide, glipizide, glyclazide; or any other insulin secretagogue such as, for example, repaglinide and nateglinide; or α-glycosidase inhibitors such as acarbose, voglibose, or miglitol. Also, the compounds of Formula I may be used in combination with HMG Co-A reductase inhibitors (statins), bile acid binding resin, or fibric acid derivatives to improve the lipid profile of subjects with dyslipidemia. Compounds of Formula I may also be used in combination with agents that regulate hypertension (e.g., inhibitors of angiotension converting enzyme (ACE), β-blockers, calcium channel blockers).

Furthermore, the compounds of this invention may have utility for the treatment of any of various CNS (central nervous system) or psychological disorders, such as the treatment of substance or behavioral addiction, and the treatment of disorders associated with the use of psychotropic substances. Likewise, the compounds of this invention may have utility for the management and treatment of cognition and memory disorders.

The compounds of Formula I may also be utilized, in free base form or in compositions, as well as in research and diagnostics or as analytical reference standards, and the like, which are well known in the art. Therefore, the present invention includes compositions which are comprised of an inert carrier and an effective amount of a compound of Formula I, or a salt, or ester thereof. An inert carrier is any material which does not interact with the compound to be carried and which lends support, means of conveyance, bulk, traceable material, and the like to the compound to be carried. An effective amount of the compound is that amount which produces a result or exerts an influence on the particular procedure being performed.

It is anticipated that prodrug forms of the compounds of this invention will prove useful in certain circumstances, and such compounds are also intended to fall within the scope of the invention. Prodrug forms may have advantages over the parent compounds exemplified herein, in that they are better absorbed, better distributed, more readily penetrate the central nervous system, are more slowly metabolized or cleared. Prodrug forms may also have formulation advantages in terms of crystallinity or water solubility. For example, compounds of the invention having one or more hydroxyl groups may be converted to esters or carbonates bearing one or more carboxyl, hydroxyl or amino groups, which are hydrolyzed at physiological pH values or are cleaved by endogenous esterases or lipases in vivo. See, for example. U.S. Pat. Nos. 4,942,184; 4,960,790; 5,817,840; and 5,824,701 (all of which are incorporated herein by reference in their entirety), and references therein.

An object of this invention is to provide a method of inducing weight loss in an individual by administration of a compound of the invention. The method of the invention comprises administering to an individual a therapeutically effective amount of at least one compound of the invention, or a prodrug thereof, which is sufficient to induce weight loss. The invention further comprises a method of preventing weight gain in an individual by administering an amount of at least one compound of the invention, or a prodrug thereof, which is sufficient to prevent weight gain.

General Preparative Methods

Compounds of Formula (I) in which R⁴═(C₁-C₆)alkyl may be prepared as shown in Reaction Scheme 1. An acylacetic ester of formula (III) is alkylated with a 2-bromoketone of formula (II) under basic conditions to give the diketo-ester of formula (IV). Suitable bases for this reaction are, for example, trialkylamines, sodium alkoxides, sodium carbonate, and the like. The pyrrole ring formation is then achieved by reaction of the formula (IV) compound with an amine of type R¹—NH₂, usually with heating, to give the pyrrole compound of formula (V). Hydrolysis of the formula (V) compound under standard conditions (for example, aqueous base or aqueous acid) gives the corresponding carboxylic acid of formula (VI). The final step is the coupling of an amine (or hydrazine) of type R⁵R⁶NH with the acid (VI) to give the compound of formula (Ia), equivalent to formula (I) where R⁴ is (C₁-C₆)alkyl. This amide-bond forming reaction may be accomplished by various methods known in the art, such as by converting the acid to its acid chloride with, for example, thionyl chloride, followed by addition of the amine in the presence of a base; or by using a coupling agent such as, for example, a carbodiimide in an inert solvent such as, for example, methylene chloride.

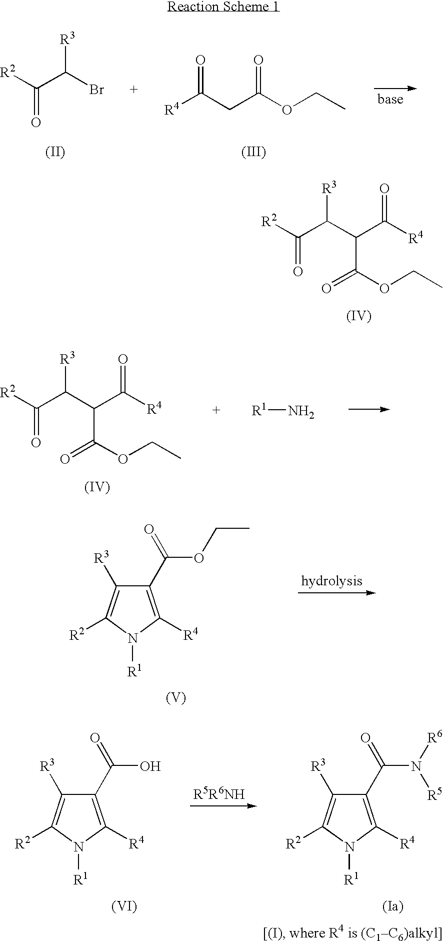

Compounds of formula (I) in which R⁴ is NH₂ may be prepared as shown in Reaction Scheme 2. Alkylation of a nitrile of formula (VII) with a 2-bromoketone (II) under basic conditions such as, for example, sodium methoxide in methanol, gives the diketone alkylation product of formula (VIII). The formula (VIII) compound is allowed to react with an amine of type R¹NH₂ under acidic conditions and with warming to produce the product of formula (Ib), equivalent to formula (I) where R⁴ is NH₂.

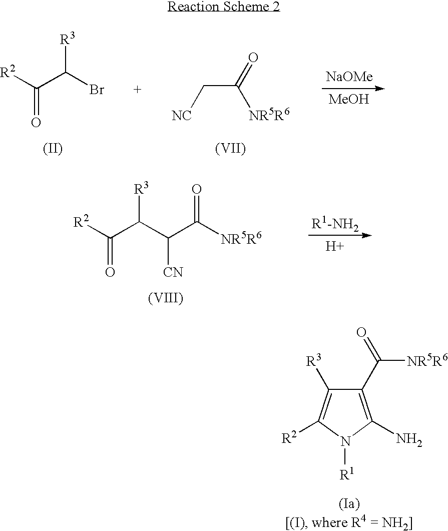

### EXPERIMENTAL EXAMPLES

The following specific preparative examples are included as illustrations of preparation of specific compounds of the invention, and are not to be construed as limiting the scope of the invention in any way.

NMR Methods:

Proton (¹H) nuclear magnetic resonance (NMR) spectra were measured with a General Electric GN-Omega 300 (300 MHz) spectrometer with either Me₄Si (δ0.00) or residual protonated solvent (CHCl₃ δ 7.26; MeOH δ 3.30; DMSO δ 2.49) as reference standard. Carbon (¹³C) NMR spectra were measured with a General Electric GN-Omega 300 (75 MHz) spectrometer with solvent (CDCl₃ δ 77.0; d₃-MeOD; δ 49.0; d₆-DMSO δ 39.5) as reference standard.

LC-MS Instrumentation:

(a) Gilson HPLC system equipped with two Gilson 306 pumps, a Gilson 215 Autosampler, a Gilson diode array detector, a YMC Pro C-18 column (2×23 mm, 120 A), and a Micromass LCZ single quadrupole mass spectrometer with z-spray electrospray ionization. Spectra were scanned from 120-800 amu over 1.5 seconds. ELSD (Evaporative Light Scattering Detector) data was also acquired as an analog channel.

(b) Hewlett-Packard 1100 HPLC equipped with a quaternary pump, a variable wavelength detector set at 254 nm, a YMC pro C-18 column (2×23 mm, 120 A), and a Finnigan LCQ ion trap mass spectrometer with electrospray ionization. Spectra were scanned from 120-1200 amu using a variable ion time according to the number of ions in the source.

HPLC Conditions:

Eluents were A: 2% acetonitrile in water with 0.02% TFA, and B: 2% water in acetonitrile with 0.02% TFA. Elution conditions consisted of a flow rate of 1.5 mL/min with an initial hold at 10% B for 0.5 minutes, followed by gradient elution from 10% B to 90% B over 3.5 minutes, followed by a final hold at 90% B for 0.5 minutes. Total run time was 4.8 minutes.

Abbreviations and Acronyms

When the following abbreviations are used herein, they have the following meaning:

conc concentrated

DMAP 4-(N,N-dimethylamino)pyidine

DMSO dimethylsulfoxide

ELSD evaporative light scattering detector

ES-MS electrospray mass spectroscopy

EtOAc ethyl acetate

EtOH ethanol (100%)

Et₃N triethylamine

h hour(s)

HPLC high performance liquid chromatography

LC-MS liquid chromatography-mass spectroscopy

min minute(s)

m/z mass-to-charge ratio

MeCN acetonitrile

PS-DIEA Polystyrene-bound diisopropylethylamine

rt room temperature

THF tetrahydrofuran

TFA trifluoroacetic acid

TFFH Fluoro-N,N,N′N′-tetramethylformamidinium hexafluorophosphate

### Example 1

### Preparation of ethyl 2-acetyl-4-(4-methoxyphenyl)4-oxobutanoate

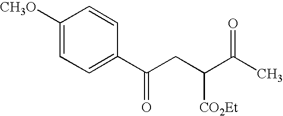

To a solution of ethyl acetoacetate (3.34 mL, 26.20 mmol) in EtOH (20 mL) at 0° C. was added dropwise sodium ethoxide (21% in EtOH, 8.49 ml, 26.20 mmol). The resulting solution was stirred for 15 minutes, then was added to a stirred solution of 2-bromo-4′-methoxyacetophenone (5 g, 21.83 mmol) in 2:1 EtOH/toluene (30 mL). The resulting mixture was stirred for 4 h at rt, then poured into 2N HCl. EtOH was removed by evaporation under reduced pressure, then EtOAc was added. The organic phase was separated, dried over MgSO₄, and evaporated. Purification by silica gel column chromatography (Biotage column # FK0-1107-17044), using 7:3 hexane/EtOAc as eluent, gave 5.12 g (83%) of ethyl 2-acetyl-4-(4-methoxyphenyl)₄-oxobutanoate as a colorless oil. ¹H NMR (400 MHz, CDCl₃) δ 7.95 (d, 2H), 6.93 (d, 2H), 4.21 (m, 3H), 3.87 (s, 3H), 3.71-3.64 (m, 1H), 3.51-3.45 (m, 1H), 2.44 (s, 3H), 1.92 (t, 3H). LC-MS m/z 279.21 (MH⁺), retention time 2.42 min.

### Example 2

### Preparation of ethyl 5-(4-methoxyphenyl)-2-methyl-1-(2-methylphenyl)-1H-pyrrole-3-carboxylate

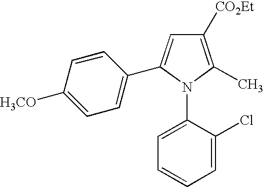

A solution of 2-chloroaniline (756 μL, 7.19 mmol) and ethyl 2-acetyl-4-(4-methoxyphenyl)-4-oxobutanoate (2 g, 7.19 mmol) in EtOH (10 mL) was heated at reflux for 5 h. The solution was cooled, evaporated under reduced pressure, and then the residue was dissolved in CH₂Cl₂. Water was added, and the organic phase was separated, dried over MgSO₄, and evaporated. The yellow oil residue was purified by silica gel column chromatography (Biotage column # FK0-1107-17044) with 7:3 to 1:1 hexane/EtOAc as solvent gradient, to give 2.48 g (95%) of ethyl 5-(4-methoxyphenyl)-2-methyl-1-(2-methylphenyl)-1H-pyrrole-3-carboxylate as a white solid. ¹H NMR (400 MHz, CDCl₃) δ 7.50-7.48 (m, 1H), 7.38-7.33 (m, 1H), 7.31-7.26 (m, 1H), 7.19-7.7.17(m, 1H), 7.02 (d, 2H), 7.00 (s, 1H), 6.69 (d, 2H), 4.31 (q, 2H), 3.73 (s, 3H), 2.32 (s, 3H), 1.38 (t, 3H). LC-MS m/z 370.23 (MH⁺), retention time 3.43 min.

### Example 3

### Preparation of 1-(2-chlorophenyl)-5-(4-methoxyphenyl-2-methyl-1H-pyrrole-3-carboxylic acid

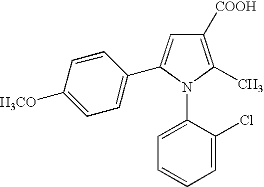

To a solution of KOH (1.1 g, 19.51 mmol) in water (16 mL) was added a solution of ethyl 5-(4-methoxyphenyl)-2-methyl-1-(2-methylphenyl)-1H-pyrrole-3-carboxylate (2.4 g, 6.5 mmol) in MeOH (16 mL). The resulting mixture was heated at reflux for 1 h, then THF (16 mL) and KOH (5 g) were added. The reaction mixture, which gradually became clear, was heated at reflux for 28 h. Solvents were evaporated, and the aqueous mixture was acidified with 2N HCl to pH 2. The mixture was extracted with EtOAc, and the combined organic extracts were dried (MgSO₄) and evaporated to give 12-chlorophenyl)-54-methoxyphenyl)-2-methyl-1H-pyrrole-3-carboxylic acid as a light yellow solid (2.15 g, 97%). ¹H NMR (400 MHz, CD₃OD) δ 7.57-7.55 (m, 1H), 7.47-7.37 (m, 2H), 7.32-7.29 (m, 1H), 7.03 (d, 2H), 6.72 (d, 2H), 6.63 (s, 1H), 3.71 (s, 3H), 2.27 (s, 3H). LC-MS m/z 342.19 (MH⁺), retention time 2.81 min.

### Example 4

### Preparation of 1-(2-chlorophenyl)-5-(4-methoxyphenyl)-2-methyl-N-(1-piperidinyl)-1H-pyrrole-3-carboxamide

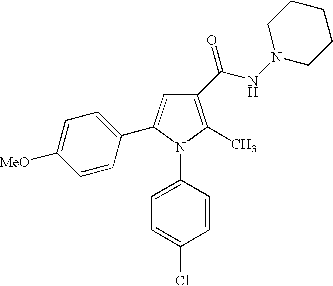

1-(2-chlorophenyl)-5-(4-methoxyphenyl)-2-methyl-1H-pyrrole-3-carboxylic acid (100 mg, 0.29 mmol), HOBT (1-hydroxybenzotriazole) (79 mg, 0.596 mmol), and EDC (1-[3-(dimethylamino)propyl]-3-ethylcarbodiimide hydrochloride) (84 mg, 0.44 mmol), were dissolved in CH₂Cl₂ (3 ml) and stirred for 1 h at rt, then 1-amino-piperidine (100 μL, 1.0 mmol) was added, followed by Et₃N (101 μL, 0.59 mmol). The resulting mixture was stirred overnight at rt, water was added, the organic phase was separated, dried over MgSO₄, and evaporated. The crude residue was purified by preparative reversed-phase HPLC, using 10 to 90% MeCN in water as gradient, to provide 20 mg (16%) of 1-(2-chlorophenyl)-5-(4-methoxyphenyl)-2-methyl-N-1-piperidinyl)-1H-pyrrole-3-carboxamide as a white solid. ¹H NMR (400 MHz, CDCl₃) δ 7.49-7.47 (m, 1H), 7.37-7.26 (m, 2H), 7.16 (bs, 1H), 7.00 (d, 2H), 6.70 (d, 2H), 6.59 (bs, 1H), 5.30 (s, 1H), 3.73 (s, 3H), 2.89 (bs, 4H), 2.33 (s, 3H), 1.77-1.75 (m, 4H), 1.45 (bs, 2H). LC-MS m/z 424.28 (MH⁺), retention time 2.40 min.

### Example 5

### Preparation of N-cyclohexylmethyl-2-methyl-1,5-diphenyl-1H-pyrrole-3-carboxamide

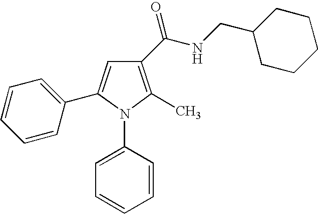

In a 8-mL screw-cap vial, 2-methyl-1,5-diphenyl-1H-pyrrole-3-carboxylic acid (28 mg, 0.1 mmol), TFFH (29 mg, 0.11 mmol) (tetramethylfluoro-formamidinium hexafluorophosphate, Advanced Chemtech, Louisville, Ky.), and 5.0 equiv. PS-DIEA (polystyrene-supported diisopropylethylamine, Argonaut Technologies Inc., San Carlos, Calif.) (loading level: 3.33 mmol/g, 150 mg, 0.5 mmol) were heated in 2 mL 1,2-dichloroethane at 35° C. overnight. The formation of acyl fluoride was monitored by LC-MS. To the mixture, 1.1 equiv. (12.5 mg, 0.11 mmol) cyclohexyl-methylamine was added and the reaction continued overnight. The mixture was filtered through a filter tube (polypropylene frit), and the filtrate was evaporated under reduced pressure. The crude product was redissolved in 1 mL MeOH and purified by preparative reversed-phase HPLC (water/acetonitrile gradient, containing 0.1% TFA) to give N-cyclohexylmethyl-2-methyl-1,5-diphenyl-1H-pyrrole-3-carboxamide as a light yellow solid (5.3 mg, 14%). ¹H NMR (400 MHz, CDCl₃) δ 7.30 (m, 3H), 7.10 (m, 5H), 7.00 (m, 2H), 6.40 (s, 1H), 5.85 (bs, 1H), 3.25 (t, 2H), 2.40 (s, 3H), 1.45-1.80 (m, 6H), 1.20 (m, 3H), 1.00 (m, 2H); LC-MS m/z 373.3 (MH⁺), retention time 4.21 min.

Summary of Examples in Table 1

Using appropriate starting materials and the experimental procedures described above for Examples 1-5, the following compounds in Table 1 were prepared. LC-MS characterization of compounds, as listed in Table 1, was carried out by using the instrumentation and methods set forth above. It will be understood by those skilled in the art that some minor modifications to the referenced procedures may have been made, but such modifications do not significantly affect the results of the preparation.

TABLE 1

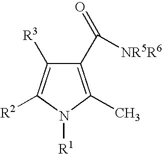

| Entry | | | | | | MS m/z | HPLC ret. |
|---|---|---|---|---|---|---|---|
| No. | R¹ | R² | R³ | NR⁵R⁶ | IUPAC name | [MH+] | time (min) |
| 1 | Ph | Ph | H | cyclohexylamino | N-cyclohexyl-2-methyl-1,5-diphenyl- | 4.02 | |
| | | | | | 1H-pyrrole-3-carboxamide | | |
| 2 | Ph | Ph | H | (4-Me-cyclohexyl)amino | 2-methyl-N-(4-methylcyclohexyl)-1,5- | 373.3 | 4.12 |
| | | | | | diphenyl-1H-pyrrale-3-carboxamide | | |
| 3 | Ph | Ph | H | cyclohexyl-methylamino | N-(cyclahexylmethyl)-2-methyl-1,5- | 373.3 | 4.21 |
| | | | | | diphenyl-1H-pyrrole-3-carboxamide | | |
| 4 | 2-Cl-Ph | 4-MeO-Ph | H | 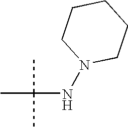 | 1-(2-chlorophenyl)-5-(4-methoxyphenyl)- 2-methyl-N-(1-piperidinyl)-1H-pyrrole-3- carboxamide hydrochloride | 424.3 | 2.40 |
| 5 | 2-Cl-Ph | 4-MeO-Ph | H | 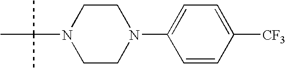 | 1-{[1-(2-chlorophenyl)-5-(4- methoxyphenyl)-2-methyl-1H-pyrrol-3- yl]carbonyl}-4-[4- (trifluoromethyl)phenyl]piperazine hydrochloride | 554.2 | 3.66 |
| 6 | 2-Cl-Ph | 4-MeO-Ph | H | cyclohexylamino | 1-(2-chlorophenyl)-N-cyclohexyl-5-(4- | 423.4 | 3.49 |
| | | | | | methoxyphenyl)-2-methyl-1H-pyrrole-3- | | |
| | | | | | carboxamide | | |
| 7 | 2-Cl-Ph | 4-MeO-Ph | H | 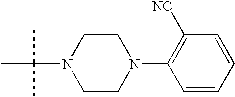 | 2-(4-{[1-(2-chlorophenyl)-5-(4- methoxyphenyl)-2-methyl-1H-pyrrol-3- yl]carbonyl}-1-piperazinyl)benzonitrile hydrochloride | 511.3 | 3.34 |
| 8 | 2-Cl-Ph | 4-MeO-Ph | H | 2-CF₃-Ph-NH—NH— | 1-(2-chlorophenyl)-5-(4-methoxyphenyl)- | 500.3 | 3.47 |
| | | | | | 2-methyl-N′-[2-(trifluoromethyl)phenyl]- | | |
| | | | | | 1H-pyrrole-3-carbohydrazide | | |
| | | | | | hydrochloride | | |
| 9 | 2-Cl-Ph | 4-MeO-Ph | H | 3-CF₃-Ph-NH—NH— | 1-(2-chlorophenyl)-5-(4-methoxyphenyl)- | 500.2 | 3.42 |
| | | | | | 2-methyl-N′-[3-(trifluoromethyl)phenyl]- | | |
| | | | | | 1H-pyrrole-3-carbohydrazide | | |
| | | | | | hydrochloride | | |
| 10 | 2-Cl-Ph | 4-MeO-Ph | H | 4-CF₃Ph-NH—NH— | 1-(2-chlorophenyl)-5-(4-methoxyphenyl)- | 500.2 | 3.39 |
| | | | | | 2-methyl-N′-[4-(trifluoromethyl)phenyl]- | | |
| | | | | | 1H-pyrrole-3-carbohydrazide | | |
| | | | | | hydrochloride | | |
| 11 | 2-Cl-Ph | 4-MeO-Ph | H | 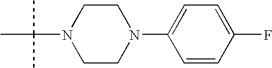 | 1-{[1-(2-chlorophenyl)-5-(4- methoxyphenyl)-2-methyl-1H-pyrrol-3- yl]carbonyl}-4-(4- fluorophenyl)piperazine hydrochloride | 504.3 | 3.50 |
| 12 | 2-Cl-Ph | 4-MeO-Ph | H | 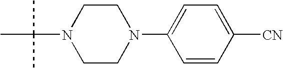 | 4-(4-{[1-(2-chlorophenyl)-5-(4- methoxyphenyl)-2-methyl-1H-pyrrol-3- yl]carbonyl}-1-piperazinyl)benzonitrile hydrocloride | 511.3 | 3.31 |
| 13 | 2-Cl-Ph | 4-MeO-Ph | H | 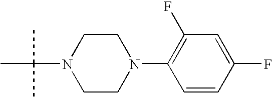 | 1-{[1-(2-chlorophenyl)-5-(4- methoxyphenyl)-2-methyl-1H-pyrrol-3- yl]carbonyl}-4-(2,4- difluorophenyl)piperazine trifluoroacetate | 522.3 | 3.48 |
| 14 | 2-Cl-Ph | 4-MeO-Ph | H | 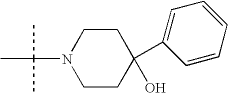 | 1-{[1-(2-chlorophenyl)-5-(4- methoxyphenyl)-2-methyl-1H-pyrrol-3- yl]carbonyl}-4-phenyl-4-piperidinol | 501.3 | 2.43 |
| 15 | 2,4-Cl₂-Ph | 4-Cl-Ph | H | 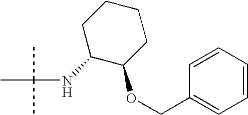 | N-[trans-2-(benzyloxy)cyclohexyl]-5-(4- chlorophenyl)-1-(2,4-dichlorophenyl)-2- methyl-1H-pyrrole-3-carboxamide | 567.2 | 3.00 |
| 16 | 2,4-Cl₂-Ph | 4-Cl-Ph | H | 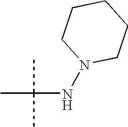 | 5-(4-chlorophenyl)-1-(2,4- dichlorophenyl)-2-methyl-N-(1- piperidinyl)-1H-pyrrole-3-carboxamide hydrochloride | 462.2 | 2.28 |
| 17 | 2,4-Cl₂-Ph | 4-Cl-Ph | H | cyclohexylamino | 5-(4-chlorophenyl)-N-cyclohexyl-1-(2,4- | 461.2 | 2.98 |
| | | | | | dichlorophenyl)-2-methyl-1H-pyrrole-3- | | |
| | | | | | carboxamide | | |
| 18 | 2,4-Cl₂-Ph | 4-Cl-Ph | H | 2-CF₃-Ph-NH—N— | 5-(4-chlorophenyl)-1-(2,4- | 538.1 | 2.93 |
| | | | | | dichlorophenyl)-2-methyl-N′-[2- | | |
| | | | | | (trifluoromethyl)phenyl]-1H-pyrrole-3- | | |
| | | | | | carbohydrazide hydrochloride | | |
| 19 | 2,4-Cl₂-Ph | 4-Cl-Ph | H | 2-CF₃-Ph-NH—NH | 5-(4-chlorophenyl)-1-(2,4- | 538.1 | 2.90 |
| | | | | | dichlorophenyl)-2-methyl-N′-[4- | | |
| | | | | | (trifluoromethyl)phenyl]-1H-pyrrole-3- | | |
| | | | | | carbohydrazide hydrochloride | | |
| 20 | 2,4-Cl₂-Ph | 4-Cl-Ph | H | 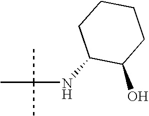 | 5-(4-chlorophenyl)-1-(2,4- dichlorophenyl)-N-[trans-2- hydroxycyclohexyl]-2-methyl-1H- pyrrole-3-carboxamide | 477.2 | 2.59 |
| 21 | 2,4-Cl₂-Ph | 4-Cl-Ph | H | 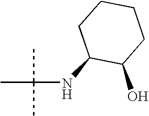 | 5-(4-chlorophenyl)-1-(2,4- dichlorophenyl)-N-[cis-2- hydroxycyclohexyl]-2-methyl-1H- pyrrole-3-carboxamide | 477.2 | 2.63 |
| 22 | 2-Cl-Ph | 4-Cl-Ph | H | 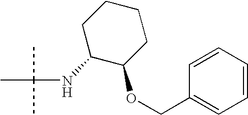 | N-[trans-2-(benzyloxy)cyclohexyl]-1-(2- chlorophenyl)-5-(4-chlorophenyl)-2- methyl-1H-pyrrole-3-carboxamide | 533.2 | 2.86 |
| 23 | 2-Cl-Ph | 4-Cl-Ph | H | 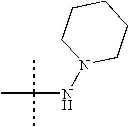 | 1-(2-chlorophenyl)-5-(4-chlorophenyl)-2- methyl-N-(1-piperidinyl)-1H-pyrrole-3- carboxamide hydrochloride | 429.3 | 2.24 |
| 24 | 2-Cl-Ph | 4-Cl-Ph | H | cyclohexylamino | 1-(2-chlorophenyl)-5-(4-chlorophenyl)-N- | 427.2 | 2.74 |
| | | | | | cyclohexyl-2-methyl-1H-pyrrole-3- | | |
| | | | | | carboxamide | | |
| 25 | 2-Cl-Ph | 4-Cl-Ph | H | 2-CF₃-Ph-NH—NH— | 1-(2-chlorophenyl)-5-(4-chlorophenyl)-2- | 504.1 | 2.79 |
| | | | | | methyl-N′-[2-(trifluoromethyl)phenyl]-1H- | | |
| | | | | | pyrrole-3-carbohydrazide hydrochloride | | |
| 26 | 2-Cl-Ph | 4-Cl-Ph | H | 4-CF₃-Ph-NH—NH— | 1-(2-chlorophenyl)-5-(4-chlorophenyl)-2- | 504.2 | 2.77 |
| | | | | | methyl-N′-[4-(trifluoromethyl)phenyl]-1H- | | |
| | | | | | pyrrole-3-carbohydrazide hydrochloride | | |
| 27 | 2-Cl-Ph | 4-Cl-Ph | H | 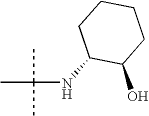 | 1-(2-chlorophenyl)-5-(4-chlorophenyl)-N- [trans-2-hydroxycyclohexyl]-2-methyl- 1H-pyrrole-3-carboxamide | 443.2 | 2.52 |
| 28 | 2-Cl-Ph | 4-Cl-Ph | H | 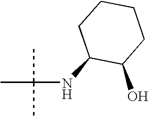 | 1-(2-chlorophenyl)-5-(4-chlorophenyl)-N- [cis-2-hydroxycyclohexyl]-2-methyl-1H- pyrrole-3-carboxamide | 443.2 | 2.58 |
| 29 | 2,4-Cl₂-Ph | 4-Cl-Ph | H | 4-Cl-benzylamino | N-(4-chlorobenzyl)-5-(4-chlorophenyl)-1- | 503.1 | 2.93 |
| | | | | | (2,4-dichlorophenyl)-2-methyl-1H- | | |
| | | | | | pyrrole-3-carboxamide | | |
| 30 | 2,4-Cl₂-Ph | 4-Cl-Ph | H | 4-F-benzylamino | 5-(4-chlorophenyl)-1-(2,4- | 487.3 | 3.78 |
| | | | | | dichlorophenyl)-N-(4-fluorobenzyl)-2- | | |
| | | | | | methyl-1H-pyrrole-3-carboxamide | | |
| 31 | 2,4-Cl₂-Ph | 4-Cl-Ph | H | 4-CF₃-benzylamino | 5-(4-chlorophenyl)-1-(2,4- | 537.1 | 3.01 |
| | | | | | dichlorophenyl)-2-methyl-N-[4- | | |
| | | | | | (trifluoromethyl)benzyl]-1H-pyrrole- | | |
| | | | | | 3-carboxamide | | |
| 32 | 2,4-Cl₂-Ph | 4-Cl-Ph | H | 4-F-Ph-NH— | 5-(4-chlorophenyl)-1-(2,4- | 473.1 | 2.98 |
| | | | | | dichlorophenyl)-N-(4-fluorophenyl)-2- | | |
| | | | | | methyl-1H-pyrrole-3-carboxamide | | |
| 33 | 2,4-Cl₂-Ph | 4-Cl-Ph | H | 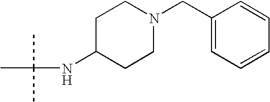 | N-(1-benzyl-4-piperidinyl)-5-(4- chlorophenyl)-1-(2,4-dichlorophenyl)-2- methyl-1H-pyrrole-3-carboxamide | 552.2 | 2.17 |
| 34 | 2,4-Cl₂-Ph | 4-Cl-Ph | H | 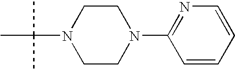 | 1-{[5-(4-chlorophenyl)-1-(2,4- dichlorophenyl)-2-methyl-1H-pyrrol-3- yl]carbonyl}-4-(2-pyridinyl)piperazine hydrochloride | 525.2 | 2.24 |
| 35 | 2,4-Cl₂-Ph | 4-Cl-Ph | H | 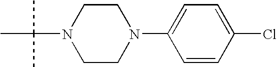 | 1-(4-chlorophenyl)-4-{[5-(4- chlorophenyl)-1-(2,4-dichlorophenyl)-2- methyl-1H-pyrrol-3- yl]carbonyl}piperazine hydrochloride | 558.1 | 3.10 |
| 36 | 2,4-Cl₂-Ph | 4-Cl-Ph | H | 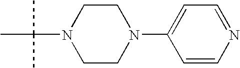 | 1-{[5-(4-chlorophenyl)-1-(2,4- dichlorophenyl)-2-methyl-1H-pyrrol-3- yl]carbonyl}-4-(4-pyridinyl)piperazine hydrochloride | 526.2 | 2.75 |
| 37 | 2,4-Cl₂-Ph | 4-Cl-Ph | H | 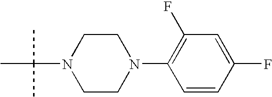 | 1-{[5-(4-chlorophenyl)-1-(2,4- dichlorophenyl)-2-methyl-1H-pyrrol-3- yl]carbonyl}-4-(2,4- difluorophenyl)piperazine hydrochloride | 560.2 | 3.07 |
| 38 | 2,4-Cl₂-Ph | 4-Cl-Ph | H | 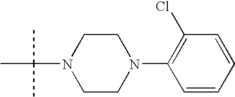 | 1-(2-chlorophenyl)-4-{[5-(4- chlorophenyl)-1-(2,4-dichlorophenyl)-2- methyl-1H-pyrrol-3- yl]carbonyl}piperazine hydrochloride | 558.1 | 3.10 |
| 39 | 2,4-Cl₂-Ph | 4-Cl-Ph | H | 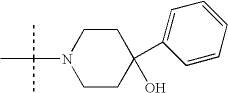 | 1-{[5-(4-chlorophenyl)-1-(2,4- dichlorophenyl)-2-methyl-1H-pyrrol-3- yl]carbonyl}-4-phenyl-4-piperidinol | 539.2 | 2.70 |
| 40 | 2,4-Cl₂-Ph | 4-Cl-Ph | H | 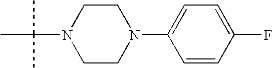 | 1-{[5-(4-chlorophenyl)-1-(2,4- dichlorophenyl)-2-methyl-1H-pyrrol-3- yl]carbonyl}-4-(4- fluorophenyl)piperazine hydrochloride | 542.2 | 2.85 |
| 41 | 2,4-Cl₂-Ph | 4-Cl-Ph | H | 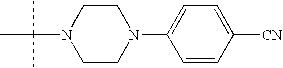 | 4-(4-{[5-(4-chlorophenyl)-1-(2,4- dichlorophenyl)-2-methyl-1H-pyrrol-3- yl]carbonyl}-1-piperazinyl)benzonitrile hydrochloride | 549.2 | 2.79 |
| 42 | 2-Cl-Ph | 4-MeO-Ph | Me | cyclohexylamino | 1-(2-chlorophenyl)-N-cyclohexyl-5-(4- | 437.3 | 3.31 |
| | | | | | methoxyphenyl)-2,4-dimethyl-1H-pyrrole- | | |
| | | | | | 3-carboxamide | | |
| 43 | 2-Cl-Ph | 4-MeO-Ph | Me | 4-CF₃-Ph-NH—NH— | 1-(2-chlorophenyl)-5-(4-methoxyphenyl)- | 514.2 | 3.45 |
| | | | | | 2,4-dimethyl-N′-[4- | | |
| | | | | | (trifluoromethyl)phenyl]-1H-pyrrole-3- | | |
| | | | | | carbohydrazide hydrochloride | | |

### Example 6

### Preparation of 2-amino-1-(2-chlorophenyl)-5-(4-chlorophenyl)-N-cyclohexyl-1H-cyclohexyl-1H-pyrrole-3-carboxamide

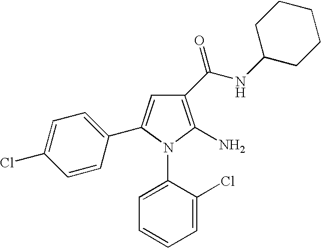

A solution of sodium methoxide (49 mg, 0.90 mmol) in methanol (5 mL) was added to a solution of 2-bromo-4′-chloroacetophenone (141 mg, 0.602 mmol) and 2-cyano-N-cyclohexyl-acetamide (100 mg, 0.602 mmol) in methanol (5 mL) under argon atmosphere over 20 minutes. The mixture was stirred at rt overnight. The methanol was evaporated off, and the residue was purified by silica gel chromatography. Elution with 5% ethyl acetate in hexane, followed by 30% ethyl acetate in hexane, gave 4-(4-chlorophenyl)-2-cyano-N-cyclohexyl-4-oxobutanamide as a white solid (103 mg, 53.5%). LC-MS m/z 319.2 (MH⁺), retention time 2.92 min.

A solution of 4-(4-chlorophenyl)-2-cyano-N-cyclohexyl-4-oxobutanamide (1.6 g, 5.0 mmol), 2-chloroaniline (529 μL, 5.0 mmol), and conc. HCl (550 μL, 5.5 mmol) in absolute ethanol was refluxed for 24 h. The ethanol was evaporated off, and the residue was purified by HPLC to give 2-amino-1-(2-chlorophenyl)-5-(4-chlorophenyl)-N-cyclohexyl-1H-pyrrole-3-carboxamide as a yellow solid (390 mg, 18%). ¹H NMR (CD₂Cl₂, 400 MHz) δ 7.45 (d, J=7.2 Hz, 1H, Ph), 7.34 (t, J=7.2 Hz, 1H, Ph), 7.29 (t, J=7.2 Hz 1H, Ph), 7.21 (d, J=7.2 Hz, 1H, Ph), 7.00 (dd, 2H, Ph), 6.88 (dd, 2H, Ph), 6.26 (s, 1H, pyrrole), 5.47 (bs, 1H, NH), 3.77 (bs, 1H, cyclohexane), 1.89 (m, 2H, cyclohexane), 1.67 (d, 1H, cyclohexane), 1.55 (d, 1H, cyclohexane), 1.31 (m, 3H, cyclohexane), 1.15 (m, 3H, cyclohexane); LC-MS m/z 428.1 (MH⁺), retention time 3.40 min.

Summary of Examples in Table 2

Using appropriate starting materials and the experimental procedures described above for Example 6, the following compounds in Table 2 were prepared. LC-MS characterization of compounds, as listed in Table 2, was carried out by using the instrumentation and methods set forth above. It will be understood by those skilled in the art that some minor modifications to the referenced procedures may have been made, but such modifications do not significantly affect the results of the preparation.

TABLE 1

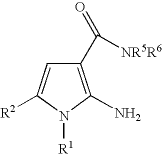

| Entry | | | | | MS m/z | HPLC ret. |
|---|---|---|---|---|---|---|
| No. | R¹ | R² | NR⁵R⁶ | IUPAC name | [MH+] | time (min) |
| 44 | 2-Cl-Ph | 4-Cl-Ph | cycloheylamino | 2-amino-1-(2-chlorophenyl)-5-(4- | 428 | 3.40 |
| | | | | chlorophenyl)-N-cyclohexyl-1H-pyrrole-3- | | |
| | | | | carboxamide hydrochloride | | |
| 45 | Ph | 4-Cl-Ph | cyclohexylamino | 2-amino-5-(4-chlorophenyl)-N-cyclohexyl-1- | 394 | 3.51 |
| | | | | phenyl-1H-pyrrole-3-carboxamide | | |
| | | | | hydrochloride | | |
| 46 | 2-F-Ph | 4-Cl-Ph | cyclohexylamino | 2-amino-5-(4-chlorophenyl)-N-cyclohexyl-1-(2- | 412 | 3.41 |
| | | | | fluorophenyl)-1H-pyrrole-3-carboxamide | | |
| | | | | hydrochloride | | |
| 47 | 2-Br-Ph | 4-Cl-Ph | cyclohexylamino | 2-amino-5-(4-chlorophenyl)-N-cyclohexyl-1-(2- | 472 | 3.51 |
| | | | | bromophenyl)-1H-pyrrole-3-carboxamide | | |
| | | | | hydrochloride | | |
| 48 | 2,4-Cl₂-Ph | 4-Cl-Ph | cyclohexylamino | 2-amino-5-(4-chlorophenyl)-N-cyclohexyl-1- | 462 | 3.65 |
| | | | | (2,4-dichlorophenyl)-1H-pyrrole-3- | | |
| | | | | carboxamide hydrochloride | | |

Summary of Examples in Table 3

Using appropriate starting materials that are known in the art or may be prepared according to methods known in the art, and using the experimental procedures described above for Example 6, the following compounds in Table 3 may be prepared.

TABLE 1

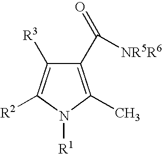

| Entry | | | | | |
|---|---|---|---|---|---|
| No. | R¹ | R² | R³ | NR⁵R⁶ | IUPAC name |
| 49 | 2-Cl-Ph | 4-Cl-Ph | CH₃ | cyclohexyl- | 2-amino-1-(2-chlorophenyl)-5-(4- |
| | | | | amino | chlorophenyl)-4-methyl-N-(1- |
| | | | | | pipendinyl)-1H-pyrrole-3-carboxamide |
| 50 | 2-Cl-Ph | 4-Cl-Ph | ethyl | cyclohexyl- | 2-amino-1-(2-chlorophenyl)-5-(4- |
| | | | | amino | chlorophenyl)-4-ethyl-N-(1- |
| | | | | | aminopiperidinyl)-1H-pyrrole-3- |
| | | | | | carboxamide |
| 51 | 2-Cl-Ph | 4-Cl-Ph | ethyl | (1-piperidinyl)- | 2-amino-1-(2-chlorophenyl)-5-(4- |
| | | | | amino | chlorophenyl)-4-ethyl-N-(1- |
| | | | | | piperidinyl)-1H-pyrrole-3- |
| | | | | | carboxamide |
| 52 | 2-Cl-Ph | 4-MeO-Ph | CH₃ | (1-piperidinyl)- | 2-amino-1-(2-chlorophenyl)-5-(4- |
| | | | | amino | methoxyphenyl)-4-methyl-N-(1- |
| | | | | | piperidinyl)-1H-pyrrole-3-carboxamide |
| 53 | 2-Cl-Ph | 4-Cl-Ph | ethyl | 4-CF₃-Ph-NH—NH | 2-amino-1-(2-chlorophenyl)-5-(4- |
| | | | | | chlorophenyl)-4-methyl-N′-[4- |
| | | | | | (trifluoromethyl)phenyl]-1H-pyrrole- |
| | | | | | 3-carbohydrazide |
| 54 | 2-Cl-Ph | 4-Cl-Ph | CH₃ | 2-CF₃-Ph-NH—NH | 2-amino-1-(2-chlorophenyl)-5-(4- |
| | | | | | chlorophenyl)-4-methyl-N′-[2- |
| | | | | | (trifluoromethyl)phenyl]-1H-pyrrole- |
| | | | | | 3-carbohydrazide |
| 55 | 2-Cl-Ph | 4-Cl-Ph | CH₃ | 4-(4- | 1-(2-chlorophenyl)-5-(4- |
| | | | | fluorobenzyl)- | chlorophenyl)-3-{[4-(4- |
| | | | | 1-piperazinyl | fluorobenzyl)-1-piperazinyl] |
| | | | | | carbonyl}-4-methyl-1H-pyrrol-2-ylamine |
| 56 | 2-Cl-Ph | 4-Cl-Ph | CH₃ | 4-(4- | 4-(4-{[2-amino-1-(2-chlorophenyl)- |
| | | | | cyanophenyl) | 5-(4-chlorophenyl)-4-methyl-1H- |
| | | | | 1-piperazinyl | pyrrol-3-yl]carbonyl}-1- |
| | | | | | piperazinyl)benzonitrile |
| 57 | 2-Cl-Ph | 4-Cl-Ph | CH₃ | trans-2- | 2-amino-N-[trans-2- |
| | | | | (benzyloxy)- | (benzyloxy)cyclohexyl]-1-(2- |
| | | | | cyclohexyl- | chlorophenyl)-5-(4-chlorophenyl)-4- |
| | | | | amino | methyl-1H-pyrrole-3-carboxamide |
| 58 | 2-Cl-Ph | 4-Cl-Ph | CH₃ | trans-2- | 2-amino-1-(2-chlorophenyl)-5-(4- |
| | | | | hydroxy- | chlorophenyl)-N-[trans-2- |
| | | | | cyclohexyl- | hydroxycyclohexyl]-4-methyl-1H- |
| | | | | amino | pyrrole-3-carboxamide |
| 59 | 2,4-Cl₂-Ph | 4-Cl-Ph | ethyl | trans-2- | 2-amino-5-(4-chlorophenyl)-1-(2,4- |
| | | | | hydroxy- | dichlorophenyl)-4-ethyl-N-[trans-2- |
| | | | | cyclopentyl- | hydroxycyclopentyl]-1H-pyrrole-3- |
| | | | | amino | carboxamide |
| 60 | 2-Cl-Ph | 4-Br-Ph | ethyl | trans-2- | 2-amino-5-(4-bromophenyl)-1-(2- |
| | | | | hydroxy- | chlorophenyl)-4-ethyl-N-[trans-2- |
| | | | | cyclohexyl- | hydroxycyclohexyl]-1H-pyrrole-3- |
| | | | | amino | carboxamide |
| 61 | 2-Cl-Ph | 4-Cl-Ph | propyl | trans-2- | 2-amino-1-(2-chlorophenyl)-5-(4- |
| | | | | hydroxy- | chlorophenyl)-N-[trans-2- |
| | | | | cyclohexyl- | hydroxycyclohexyl]-4-propyl-1H- |
| | | | | amino | pyrrole-3-carboxamide |

### Evaluation of Biological Activity

Evaluation of Compound's Efficacy on the Reduction of Food Intake (Suppression of Appetite) in Lean Overnight Fasted Rats

Fasted-Refed Acute Feeding Assay

The purpose of this protocol is to determine the effect of a single dose of an unknown compound on food consumption of lean overnight fasted rats. The fasted-refed rat model is frequently used in the field of obesity to identify compounds with potential for anorectic effects. This animal model has been successfully used in the identification and characterization of the efficacy profile of compounds that are or have been used in the management of body weight in obese humans (see, e.g., Balvet et al., Gen. Pharmacol. 13:293-297, 1982; Grignaschi et al., Br. J. Pharmacol. 127:1190-1194, 1999; McTavish and Heel, Drug 43:713-733, 1992; Rowland et al., Life Sci. 36:2295-2300, 1985).

A typical study includes 60-80 male rats (n=10/treatment group) with an average body weight of approximately 280 g. Rats are kept in standard animal rooms under controlled temperature and humidity and a 12/12 light dark cycle. Rats are single-housed in suspended cages with a mesh floor. Water and food are continuously available unless the animals are being fasted for the study.

The vehicle test: The rats are grouped based upon their performance on a vehicle test. The vehicle test is performed between 2 and 7 days before the efficacy test. The rats are fasted overnight during the dark phase (total of approx. 16-18 hrs). The animal is dosed with 0.5 mL deionized water. One hour after dosing, pre-weighed food jars are returned to the animal home cage. The rats are allowed one hour of feeding time. After 1 hour, the spillage is returned to the food jar and the amount of food consumed is determined. The rats are assigned to groups so that the mean and standard error of the mean of 1-hour food consumption are similar between groups.

The efficacy test: The rats are fasted overnight during the dark phase (total of approx. 16-18 hr). The animal is dosed with an assigned treatment (2 mg/ml). One hour after dosing, pre-weighed food jars are returned to the cage. Food intake is recorded 30, 60, 90, 180, and 240 minutes post-food return. At each time point, spillage is returned to the food jar and then the food jars are weighed. The amount of food consumed is determined for each time point. Difference between treatment group is determined using appropriate statistical analysis.

Compounds of this invention were found to be active in this fasted-refed acute feeding assay. For example, when the pyrrole derivative 1-(2-chlorophenyl)-5-(4-chlorophenyl)-2-methyl-N-(1-piperidinyl)-1H-pyrrole-3-carboxamide hydrochloride (Table 1, entry 23) was dosed at 10 mg/kg p.o., food consumption was reduced (relative to the food consumption observed for the vehicle control group) by up to 25% when measured at time points from 30 to 240 minutes. Likewise, when the pyrrole derivative 1-(2-chlorophenyl)-5-(4-methoxyphenyl)-2,4-dimethyl-N′-[4-(trifluoromethyl)phenyl]-1H-pyrrole-3-carbohydrazide hydrochloride (Table 1, entry 43) was dosed at 10 mg/kg p.o., food consumption was reduced (relative to the food consumption observed for the vehicle control group) by up to 35% when measured at time points from 30 to 240 minutes.

Evaluation of Compound's Efficacy on the Reduction of Body Weight and Food and Water Consumption in Obese Zucker fa/fa Rats

Chronic Feeding Assay

The purpose of this protocol is to determine the effect of chronic administration of an unknown compound on body weight and food and water consumption in obese Zucker fa/fa rats. Obese Zucker fa/fa rats are frequently used in the determination of compound efficacy in the reduction of body weight. This animal model has been successfully used in the identification and characterization of the efficacy profile of compounds that are or have been used in the management of body weight in obese humans (see, e.g., A1-Barazanji et al., Obes Res. 8:317-323, 2000; Assimacopoulos-Jeannet et al., Am. J. Physiol. 260(2 Pt 2):R278-283, 1991; Dryden et al., Horm. Metab. Res. 31:363-366, 1999; Edwards and Stevens, Pharmacol. Biochem. Behav. 47:865-872, 1994; Grinker et al., Pharmacol. Biochem. Behav. 12:265-275, 1980).

A typical study includes 60-80 male Zucker fa/fa (n=10/treatment group) with an average body weight of approximately 550 g. Rats are kept in standard animal rooms under controlled temperature and humidity and a 12/12 light dark cycle. Water and food are continuously available. Rats are single-housed in large rat shoeboxes containing grid floor. Animals are adapted to the grid floors and sham-dosed with study vehicle for at least four days before the recording of two-days baseline measurement of body weight and 24-hr food and water consumption. Rats are assigned to one of 6-8 treatment groups based upon their body weight on baseline. The groups are set up so that the mean and standard error of the mean of body weight were similar.

Animals are orally gavaged (2 mL/kg) daily before the dark phase of the LD/cycle for a pre-determined number of days (typically 6-14 days) with their assigned dose/compound. At this time, body weight, food and water consumption are measured. On the final day, animals are euthanized by CO₂ inhalation, and the body weight is measured.

The efficacy of compounds of this invention on the reduction or control of body weight may be determined by using this chronic feeding assay.

Measurement of Brain Exposure

Male obese Zucker fa/fa rats are administered compounds, typically at 10 mg/kg p.o., and then brains are collected at 2 hours post dosing for determination of brain concentration. Brains are weighed and homogenized with 4 mL of 10 mM ammonium acetate buffer (pH 3), and the brain tissue homogenate samples are extracted via protein precipitation with acetonitrile. Samples are vortexed, centrifuged and analyzed by liquid chromatography utilizing mass spectrometer selective detection (LC/MS/MS) using the heated nebulizer interface. Samples are quantitated using weighted (1/x²) linear internal standard calibration curve.

The level of brain exposure of the compounds of this invention may be determined by using this assay.

Demonstration of the activity of the compounds of the present invention may be accomplished through in vitro, ex vivo, and in vivo assays that are well known in the art. For example, to demonstrate the efficacy of a pharmaceutical agent for the treatment of diabetes and related disorders such as Syndrome X, impaired glucose tolerance, impaired fasting glucose, and hyperinsulinemia or atherosclerotic disease and related disorders such as hypertriglyceridemia and hypercholesteremia, the following assays may be used.

Method for Measuring Blood Glucose Levels

db/db mice (obtained from Jackson Laboratories, Bar Harbor, Me.) are bled (by either eye or tail vein) and grouped according to equivalent mean blood glucose levels. They are dosed orally (by gavage in a pharmaceutically acceptable vehicle) with the test compound once daily for 14 days. At this point, the animals are bled again by eye or tail vein and blood glucose levels were determined. In each case, glucose levels are measured with a Glucometer Elite XL (Bayer Corporation, Elkhart, Ind.).

Method for Measuring Triglyceride Levels

hApoA1 mice (obtained from Jackson Laboratories, Bar Harbor, Me.) are bled (by either eye or tail vein) and grouped according to equivalent mean serum triglyceride levels. They are dosed orally (by gavage in a pharmaceutically acceptable vehicle) with the test compound once daily for 8 days. The animals are then bled again by eye or tail vein, and serum triglyceride levels are determined. In each case, triglyceride levels are measured using a Technicon Axon Autoanalyzer (Bayer Corporation, Tarrytown, N.Y.).

Method for Measuring HDL-Cholesterol Levels

To determine plasma HDL-cholesterol levels, hApoA1 mice are bled and grouped with equivalent mean plasma HDL-cholesterol levels. The mice are orally dosed once daily with vehicle or test compound for 7 days, and then bled again on day 8. Plasma is analyzed for HDL cholesterol using the Synchron Clinical System (CX4) (Beckman Coulter, Fullerton, Calif.).

Method for Measuring Total Cholesterol, HDL-Cholesterol, Triglycerides, and Glucose Levels

In another in vivo assay, obese monkeys are bled, then orally dosed once daily with vehicle or test compound for 4 weeks, and then bled again. Serum is analyzed for total cholesterol, HDL-cholesterol, triglycerides, and glucose using the Synchron Clinical System (CX4) (Beckman Coulter, Fullerton, Calif.). Lipoprotein subclass analysis is performed by NMR spectroscopy as described by Oliver et al., (Proc. Natl. Acad. Sci. USA 98:5306-5311, 2001).

Method for Measuring an Effect on Cardiovascular Parameters

Cardiovascular parameters (e.g., heart rate and blood pressure) are also evaluated. SHR rats are orally dosed once daily with vehicle or test compound for 2 weeks. Blood pressure and heart rate are determined using a tail-cuff method as described by Grinsell et al., (Am. J. Hypertens. 13:370-375, 2000). In monkeys, blood pressure and heart rate are monitored as described by Shen et al., (J. Pharmacol. Exp. Therap. 278:1435-1443, 1996).

Pharmaceutical Compositions

Based on the above tests, or other well known assays used to determine the efficacy for treatment of conditions identified above in mammals, and by comparison of these results with the results of known medicaments that are used to treat these conditions, the effective dosage of the compounds of this invention can readily be determined for treatment of each desired indication. The amount of the active ingredient to be administered in the treatment of one of these conditions can vary widely according to such considerations as the particular compound and dosage unit employed, the mode of administration, the period of treatment, the age and sex of the patient treated, and the nature and extent of the condition treated.

The total amount of the active ingredient to be administered may generally range from about 0.001 mg/kg to about 200 mg/kg, and preferably from about 0.01 mg/kg to about 200 mg/kg body weight per day. A unit dosage may contain from about 0.05 mg to about 1500 mg of active ingredient, and may be administered one or more times per day. The daily dosage for administration by injection, including intravenous, intramuscular, subcutaneous, and parenteral injections, and use of infusion techniques may be from about 0.01 to about 200 mg/kg. The daily rectal dosage regimen may be from 0.01 to 200 mg/kg of total body weight. The transdermal concentration may be that required to maintain a daily dose of from 0.01 to 200 mg/kg.

Of course, the specific initial and continuing dosage regimen for each patient will vary according to the nature and severity of the condition as determined by the attending diagnostician, the activity of the specific compound employed, the age of the patient, the diet of the patient, time of administration, route of administration, rate of excretion of the drug, drug combinations, and the like. The desired mode of treatment and number of doses of a compound of the present invention or a pharmaceutically acceptable salt thereof may be ascertained by those skilled in the art using conventional treatment tests.

The compounds of this invention may be utilized to achieve the desired pharmacological effect by administration to a patient in need thereof in an appropriately formulated pharmaceutical composition. A patient, for the purpose of this invention, is a mammal, including a human, in need of treatment for a particular condition or disease. Therefore, the present invention includes pharmaceutical compositions which are comprised of a pharmaceutically acceptable carrier and a pharmaceutically effective amount of a compound identified by the methods described herein, or a pharmaceutically acceptable salt or ester thereof. A pharmaceutically acceptable carrier is any carrier which is relatively non-toxic and innocuous to a patient at concentrations consistent with effective activity of the active ingredient so that any side effects ascribable to the carrier do not vitiate the beneficial effects of the active ingredient. A pharmaceutically effective amount of a compound is that amount which produces a result or exerts an influence on the particular condition being treated. The compounds identified by the methods described herein may be administered with a pharmaceutically-acceptable carrier using any effective conventional dosage unit forms, including, for example, immediate and timed release preparations, orally, parenterally, topically, or the like.

For oral administration, the compounds may be formulated into solid or liquid preparations such as, for example, capsules, pills, tablets, troches, lozenges, melts, powders, solutions, suspensions, or emulsions, and may be prepared according to methods known to the art for the manufacture of pharmaceutical compositions. The solid unit dosage forms may be a capsule which can be of the ordinary hard- or soft-shelled gelatin type containing, for example, surfactants, lubricants, and inert fillers such as lactose, sucrose, calcium phosphate, and corn starch.

In another embodiment, the compounds of this invention may be tableted with conventional tablet bases such as lactose, sucrose, and cornstarch in combination with binders such as acacia, cornstarch, or gelatin; disintegrating agents intended to assist the break-up and dissolution of the tablet following administration such as potato starch, alginic acid, corn starch, and guar gum; lubricants intended to improve the flow of tablet granulation and to prevent the adhesion of tablet material to the surfaces of the tablet dies and punches, for example, talc, stearic acid, or magnesium, calcium or zinc stearate; dyes; coloring agents; and flavoring agents intended to enhance the aesthetic qualities of the tablets and make them more acceptable to the patient. Suitable excipients for use in oral liquid dosage forms include diluents such as water and alcohols, for example, ethanol, benzyl alcohol, and polyethylene alcohols, either with or without the addition of a pharmaceutically acceptable surfactant, suspending agent, or emulsifying agent. Various other materials may be present as coatings or to otherwise modify the physical form of the dosage unit. For instance tablets, pills or capsules may be coated with shellac, sugar or both.

Dispersible powders and granules are suitable for the preparation of an aqueous suspension. They provide the active ingredient in admixture with a dispersing or wetting agent, a suspending agent, and one or more preservatives. Suitable dispersing or wetting agents and suspending agents are exemplified by those already mentioned above. Additional excipients, for example, those sweetening, flavoring and coloring agents described above, may also be present.

The pharmaceutical compositions of this invention may also be in the form of oil-in-water emulsions. The oily phase may be a vegetable oil such as liquid paraffin or a mixture of vegetable oils. Suitable emulsifying agents may be (1) naturally occurring gums such as gum acacia and gum tragacanth, (2) naturally occurring phosphatides such as soy bean and lecithin, (3) esters or partial esters derived from fatty acids and hexitol anhydrides, for example, sorbitan monooleate, and (4) condensation products of said partial esters with ethylene oxide, for example, polyoxyethylene sorbitan monooleate. The emulsions may also contain sweetening and flavoring agents.

Oily suspensions may be formulated by suspending the active ingredient in a vegetable oil such as, for example, arachis oil, olive oil, sesame oil, or coconut oil; or in a mineral oil such as liquid paraffin. The oily suspensions may contain a thickening agent such as, for example, beeswax, hard paraffin, or cetyl alcohol. The suspensions may also contain one or more preservatives, for example, ethyl or n-propyl p-hydroxybenzoate; one or more coloring agents; one or more flavoring agents; and one or more sweetening agents such as sucrose or saccharin.

Syrups and elixirs may be formulated with sweetening agents such as, for example, glycerol, propylene glycol, sorbitol, or sucrose. Such formulations may also contain a demulcent, and preservative, flavoring and coloring agents.

The compounds of this invention may also be administered parenterally, that is, subcutaneously, intravenously, intramuscularly, or interperitoneally, as injectable dosages of the compound in a physiologically acceptable diluent with a pharmaceutical carrier which may be a sterile liquid or mixture of liquids such as water, saline, aqueous dextrose and related sugar solutions; an alcohol such as ethanol, isopropanol, or hexadecyl alcohol; glycols such as propylene glycol or polyethylene glycol; glycerol ketals such as 2,2-dimethyl-1,1-dioxolane-4-methanol, ethers such as poly(ethyleneglycol) 400; an oil; a fatty acid; a fatty acid ester or glyceride; or an acetylated fatty acid glyceride with or without the addition of a pharmaceutically acceptable surfactant such as a soap or a detergent, suspending agent such as pectin, carbomers, methycellulose, hydroxypropylmethylcellulose, or carboxymethylcellulose, or emulsifying agent and other pharmaceutical adjuvants.

Illustrative of oils which can be used in the parenteral formulations of this invention are those of petroleum, animal, vegetable, or synthetic origin, for example, peanut oil, soybean oil, sesame oil, cottonseed oil, corn oil, olive oil, petrolatum, and mineral oil. Suitable fatty acids include oleic acid, stearic acid, and isostearic acid. Suitable fatty acid esters are, for example, ethyl oleate and isopropyl myristate. Suitable soaps include fatty alkali metal, ammonium, and triethanolamine salts and suitable detergents include cationic detergents, for example, dimethyl dialkyl ammonium halides, alkyl pyridinium halides, and alkylamine acetates; anionic detergents, for example, alkyl, aryl, and olefin sulfonates, alkyl, olefin, ether, and monoglyceride sulfates, and sulfosuccinates; nonionic detergents, for example, fatty amine oxides, fatty acid alkanolamides, and polyoxyethylenepolypropylene copolymers; and amphoteric detergents, for example, alkyl-beta-aminopropionates, and 2-alkylimidazoline quarternary ammonium salts, as well as mixtures.

The parenteral compositions of this invention may typically contain from about 0.5% to about 25% by weight of the active ingredient in solution. Preservatives and buffers may also be used advantageously. In order to minimize or eliminate irritation at the site of injection, such compositions may contain a non-ionic surfactant having a hydrophile-lipophile balance (HLB) of from about 12 to about 17. The quantity of surfactant in such formulation ranges from about 5% to about 15% by weight. The surfactant can be a single component having the above HLB or can be a mixture of two or more components having the desired HLB.

Illustrative of surfactants used in parenteral formulations are the class of polyethylene sorbitan fatty acid esters, for example, sorbitan monooleate and the high molecular weight adducts of ethylene oxide with a hydrophobic base, formed by the condensation of propylene oxide with propylene glycol.

The pharmaceutical compositions may be in the form of sterile injectable aqueous suspensions. Such suspensions may be formulated according to known methods using suitable dispersing or wetting agents and suspending agents such as, for example, sodium carboxymethylcellulose, methylcellulose, hydroxypropylmethyl-cellulose, sodium alginate, polyvinylpyrrolidone, gum tragacanth and gum acacia; dispersing or wetting agents which may be a naturally occurring phosphatide such as lecithin, a condensation product of an alkylene oxide with a fatty acid, for example, polyoxyethylene stearate, a condensation product of ethylene oxide with a long chain aliphatic alcohol, for example, heptadecaethyleneoxycetanol, a condensation product of ethylene oxide with a partial ester derived form a fatty acid and a hexitol such as polyoxyethylene sorbitol monooleate, or a condensation product of an ethylene oxide with a partial ester derived from a fatty acid and a hexitol anhydride, for example polyoxyethylene sorbitan monooleate.

The sterile injectable preparation may also be a sterile injectable solution or suspension in a non-toxic parenterally acceptable diluent or solvent. Diluents and solvents that may be employed are, for example, water, Ringer's solution, and isotonic sodium chloride solution. In addition, sterile fixed oils are conventionally employed as solvents or suspending media. For this purpose, any bland, fixed oil may be employed including synthetic mono or diglycerides. In addition, fatty acids such as oleic acid may be used in the preparation of injectables.

A composition of the invention may also be administered in the form of suppositories for rectal administration of the drug. These compositions may be prepared by mixing the drug with a suitable non-irritation excipient which is solid at ordinary temperatures but liquid at the rectal temperature and will therefore melt in the rectum to release the drug. Such material are, for example, cocoa butter and polyethylene glycol.

Another formulation employed in the methods of the present invention employs transdermal delivery devices (“patches”). Such transdermal patches may be used to provide continuous or discontinuous infusion of the compounds of the present invention in controlled amounts. The construction and use of transdermal patches for the delivery of pharmaceutical agents is well known in the art (see, e.g., U.S. Pat. No. 5,023,252, incorporated herein by reference). Such patches may be constructed for continuous, pulsatile, or on demand delivery of pharmaceutical agents.

It may be desirable or necessary to introduce the pharmaceutical composition to the patient via a mechanical delivery device. The construction and use of mechanical delivery devices for the delivery of pharmaceutical agents is well known in the art. For example, direct techniques for administering a drug directly to the brain usually involve placement of a drug delivery catheter into the patient's ventricular system to bypass the blood-brain barrier. One such implantable delivery system, used for the transport of agents to specific anatomical regions of the body, is described in U.S. Pat. No. 5,011,472, incorporated herein by reference.

The compositions of the invention may also contain other conventional pharmaceutically acceptable compounding ingredients, generally referred to as carriers or diluents, as necessary or desired. Any of the compositions of this invention may be preserved by the addition of an antioxidant such as ascorbic acid or by other suitable preservatives. Conventional procedures for preparing such compositions in appropriate dosage forms can be utilized.

Commonly used pharmaceutical ingredients which may be used as appropriate to formulate the composition for its intended route of administration include: acidifying agents, for example, but are not limited to, acetic acid, citric acid, fumaric acid, hydrochloric acid, nitric acid; and alkalinizing agents such as, but are not limited to, ammonia solution, ammonium carbonate, diethanolamine, monoethanolamine, potassium hydroxide, sodium borate, sodium carbonate, sodium hydroxide, triethanolamine, trolamine.

Other pharmaceutical ingredients include, for example, but are not limited to, adsorbents (e.g., powdered cellulose and activated charcoal); aerosol propellants (e.g., carbon dioxide, CCl₂F₂, F₂ClC—CClF₂ and CClF₃); air displacement agents (e.g., nitrogen and argon); antifungal preservatives (e.g., benzoic acid, butylparaben, ethylparaben, methylparaben, propylparaben, sodium benzoate); antimicrobial preservatives (e.g., benzalkonium chloride, benzethonium chloride, benzyl alcohol, cetylpyridinium chloride, chlorobutanol, phenol, phenylethyl alcohol, phenylmercuric nitrate and thimerosal); antioxidants (e.g., ascorbic acid, ascorbyl palmitate, butylated hydroxyanisole, butylated hydroxytoluene, hypophosphorus acid, monothioglycerol, propyl gallate, sodium ascorbate, sodium bisulfite, sodium formaldehyde sulfoxylate, sodium metabisulfite); binding materials (e.g., block polymers, natural and synthetic rubber, polyacrylates, polyurethanes, silicones and styrene-butadiene copolymers); buffering agents (e.g., potassium metaphosphate, potassium phosphate monobasic, sodium acetate, sodium citrate anhydrous and sodium citrate dihydrate); carrying agents (e.g., acacia syrup, aromatic syrup, aromatic elixir, cherry syrup, cocoa syrup, orange syrup, syrup, corn oil, mineral oil, peanut oil, sesame oil, bacteriostatic sodium chloride injection and bacteriostatic water for injection); chelating agents (e.g., edetate disodium and edetic acid); colorants (e.g., FD&C Red No. 3, FD&C Red No. 20, FD&C Yellow No. 6, FD&C Blue No. 2, D&C Green No. 5, D&C Orange No. 5, D&C Red No. 8, caramel and ferric oxide red); clarifying agents (e.g., bentonite); emulsifying agents (but are not limited to, acacia, cetomacrogol, cetyl alcohol, glyceryl monostearate, lecithin, sorbitan monooleate, polyethylene 50 stearate); encapsulating agents (e.g., gelatin and cellulose acetate phthalate); flavorants (e.g., anise oil, cinnamon oil, cocoa, menthol, orange oil, peppermint oil and vanillin); humectants (e.g., glycerin, propylene glycol and sorbitol); levigating agents (e.g., mineral oil and glycerin); oils (e.g., arachis oil, mineral oil, olive oil, peanut oil, sesame oil and vegetable oil); ointment bases (e.g., lanolin, hydrophilic ointment, polyethylene glycol ointment, petrolatum, hydrophilic petrolatum, white ointment, yellow ointment, and rose water ointment); penetration enhancers (transdermal delivery) (e.g., monohydroxy or polyhydroxy alcohols, saturated or unsaturated fatty alcohols, saturated or unsaturated fatty esters, saturated or unsaturated dicarboxylic acids, essential oils, phosphatidyl derivatives, cephalin, terpenes, amides, ethers, ketones and ureas); plasticizers (e.g., diethyl phthalate and glycerin); solvents (e.g., alcohol, corn oil, cottonseed oil, glycerin, isopropyl alcohol, mineral oil, oleic acid, peanut oil, purified water, water for injection, sterile water for injection and sterile water for irrigation); stiffening agents (e.g., cetyl alcohol, cetyl esters wax, microcrystalline wax, paraffin, stearyl alcohol, white wax and yellow wax); suppository bases (e.g., cocoa butter and polyethylene glycols (mixtures)); surfactants (e.g., benzalkonium chloride, nonoxynol 10, oxtoxynol 9, polysorbate 80, sodium lauryl sulfate and sorbitan monopalmitate); suspending agents (e.g., agar, bentonite, carbomers, carboxymethylcellulose sodium, hydroxyethyl cellulose, hydroxypropyl cellulose, hydroxypropyl methylcellulose, kaolin, methylcellulose, tragacanth and veegum); sweetening e.g., aspartame, dextrose, glycerin, mannitol, propylene glycol, saccharin sodium, sorbitol and sucrose); tablet anti-adherents (e.g., magnesium stearate and talc); tablet binders (e.g., acacia, alginic acid, carboxymethylcellulose sodium, compressible sugar, ethylcellulose, gelatin, liquid glucose, methylcellulose, povidone and pregelatinized starch); tablet and capsule diluents (e.g., dibasic calcium phosphate, kaolin, lactose, mannitol, microcrystalline cellulose, powdered cellulose, precipitated calcium carbonate, sodium carbonate, sodium phosphate, sorbitol and starch); tablet coating agents (e.g., liquid glucose, hydroxyethyl cellulose, hydroxypropyl cellulose, hydroxypropyl methylcellulose, methylcellulose, ethylcellulose, cellulose acetate phthalate and shellac); tablet direct compression excipients (e.g., dibasic calcium phosphate); tablet disintegrants (e.g., alginic acid, carboxymethylcellulose calcium, microcrystalline cellulose, polacrillin potassium, sodium alginate, sodium starch glycollate and starch); tablet glidants (e.g., colloidal silica, corn starch and talc); tablet lubricants (e.g., calcium stearate, magnesium stearate, mineral oil, stearic acid and zinc stearate); tablet/capsule opaquants (e.g., titanium dioxide); tablet polishing agents (e.g., carnuba wax and white wax); thickening agents (e.g., beeswax, cetyl alcohol and paraffin); tonicity agents (e.g., dextrose and sodium chloride); viscosity increasing agents (e.g., alginic acid, bentonite, carbomers, carboxymethylcellulose sodium, methylcellulose, povidone, sodium alginate and tragacanth); and wetting agents (e.g., heptadecaethylene oxycetanol, lecithins, polyethylene sorbitol monooleate, polyoxyethylene sorbitol monooleate, and polyoxyethylene stearate).

The compounds identified by the methods described herein may be administered as the sole pharmaceutical agent or in combination with one or more other pharmaceutical agents where the combination causes no unacceptable adverse effects. For example, the compounds of this invention can be combined with known anti-obesity, or with known antidiabetic or other indication agents, and the like, as well as with admixtures and combinations thereof.

The compounds identified by the methods described herein may also be utilized, in free base form or in compositions, in research and diagnostics, or as analytical reference standards, and the like. Therefore, the present invention includes compositions which are comprised of an inert carrier and an effective amount of a compound identified by the methods described herein, or a salt or ester thereof. An inert carrier is any material which does not interact with the compound to be carried and which lends support, means of conveyance, bulk, traceable material, and the like to the compound to be carried. An effective amount of compound is that amount which produces a result or exerts an influence on the particular procedure being performed.

Formulations suitable for subcutaneous, intravenous, intramuscular, and the like; suitable pharmaceutical carriers; and techniques for formulation and administration may be prepared by any of the methods well known in the art (see, e.g., Remington's Pharmaceutical Sciences, Mack Publishing Co., Easton, Pa., 20^{th} edition, 2000) The following examples are presented to illustrate the invention described herein, but should not be construed as limiting the scope of the invention in any way.

Capsule Formulation

| | |
|---|---|
| A capsule formula is prepared from: | |
| Compound of this invention | 40 mg |
| Starch | 109 mg |
| Magnesium stearate | 1 mg |

The components are blended, passed through an appropriate mesh sieve, and filled into hard gelatin capsules.

Tablet Formulation

| | |
|---|---|
| A tablet is prepared from: | |
| Compound of this invention | 25 mg |
| Cellulose, microcrystaline | 200 mg |
| Colloidal silicon dioxide | 10 mg |
| Stearic acid | 5.0 mg |

The ingredients are mixed and compressed to form tablets. Appropriate aqueous and non-aqueous coatings may be applied to increase palatability, improve elegance and stability or delay absorption.

Sterile IV Solution

A 5 mg/ml solution of the desired compound of this invention is made using sterile, injectable water, and the pH is adjusted if necessary. The solution is diluted for administration to 1-2 mg/ml with sterile 5% dextrose and is administered as an IV infusion over 60 minutes.

Intramuscular Suspension

The following intramuscular suspension is prepared:

| | |
|---|---|
| Compound of this invention | 50 mg/ml |
| Sodium carboxymethylcellulose | 5 mg/ml |
| TWEEN 80 | 4 mg/ml |
| Sodium chloride | 9 mg/ml |
| Benzyl alcohol | 9 mg/ml |

The suspension is administered intramuscularly.

Hard Shell Capsules

A large number of unit capsules are prepared by filling standard two-piece hard galantine capsules each with 100 mg of powdered active ingredient, 150 mg of lactose, 50 mg of cellulose and 6 mg of magnesium stearate.

Soft Gelatin Capsules

A mixture of active ingredient in a digestible oil such as soybean oil, cottonseed oil or olive oil is prepared and injected by means of a positive displacement pump into molten gelatin to form soft gelatin capsules containing 100 mg of the active ingredient. The capsules are washed and dried. The active ingredient can be dissolved in a mixture of polyethylene glycol, glycerin and sorbitol to prepare a water miscible medicine mix.

Immediate Release Tablets/Capsules

These are solid oral dosage forms made by conventional and novel processes. These units are taken orally without water for immediate dissolution and delivery of the medication. The active ingredient is mixed in a liquid containing ingredient such as sugar, gelatin, pectin and sweeteners. These liquids are solidified into solid tablets or caplets by freeze drying and solid state extraction techniques. The drug compounds may be compressed with viscoelastic and thermoelastic sugars and polymers or effervescent components to produce porous matrices intended for immediate release, without the need of water.

The structures, materials, compositions, and methods described herein are intended to be representative examples of the invention, and it will be understood that the scope of the invention is not limited by the scope of the examples. Those skilled in the art will recognize that the invention may be practiced with variations on the disclosed structures, materials, compositions and methods, and such variations are regarded as within the ambit of the invention.

We claim:

1. A compound of Formula (I) 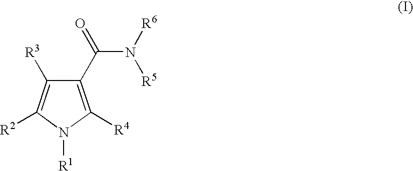
wherein
R¹ and R² are each a phenyl group, optionally substituted with one or more halogen, (C₁-C₆)alkyl, (C₁-C₆)alkoxy, trifluoromethyl, hydroxy, cyano, or nitro;
R³ is hydrogen;
R⁴ is CH₃;
R⁵ is hydrogen or (C₁-C₆)alkyl;
R⁶ is cyclohexyl which is substituted with one or more (C₁-C₃)alkyl, hydroxy, benzyloxy, (C₁-C₆)alkoxy, (C₁-C₆)alkyl-amino, bis[(C₁-C₃)alkyl]-amino, or fluorine,
(C₁-C₅)alkyl, optionally substituted with one or more cyclo(C₃-C₇)alkyl, hydroxy, benzyloxy, (C₁-C₆)alkoxy, (C₁-C₆)alkyl-amino, bis[(C₁-C₃)alkyl]-amino, or fluorine,
cyclopentyl, cycloheptyl or cyclo(C₃-C₇)alkyl-(C₁-C₃)alkyl, each of which may be optionally substituted with one or more (C₁-C₃)alkyl, hydroxy, benzyloxy, (C₁-C₆)alkoxy, (C₁-C₆)alkyl-amino, bis[(C₁-C₃)alkyl]-amino, or fluorine,
benzyl which is substituted on the phenyl ring with one or more fluorine, bromine, (C₁-C₆)alkyl, (C₁-C₆)alkoxy, trifluoromethyl, cyano, hydroxy, benzyloxy, or nitro,
phenyl substituted with one or more (C₁-C₆)alkyl, (C₁-C₆)alkoxy, trifluoromethyl, cyano, hydroxy, benzyloxy, nitro, or halogen,
piperidin-4-yl, piperidin-3-yl, or pyrrolidin-3-yl, each of which may be optionally substituted on the nitrogen atom of the piperidine or pyrrolidine ring with (C₁-C₆)alkyl, hydroxy-substituted (C₁-C₆)alkyl, or a benzyl or phenyl group that is optionally substituted on the phenyl ring with one or more of (C₁-C₆)alkyl, (C₁-C₆)alkoxy, trifluoromethyl, cyano, hydroxy, or halogen,
—NR⁷R⁸
where R⁷ is hydrogen or (C₁-C₆)alkyl;
R⁸ is (C₁-C₉)alkyl, or a phenyl group that is optionally substituted with one or more (C₁-C₆)alkyl, (C₁₋₆)alkoxy, hydroxy-substituted (C₁₋₆)alkyl, hydroxy, trifluoromethyl, cyano, nitro, or halogen; or
R⁷ and R⁸, taken together with the nitrogen atom to which they are attached, form a 5- to 10-membered saturated heterocyclic radical which is optionally substituted by one or more (C₁-C₆)alkyl, (C₁-C₆)alkoxy, hydroxy-substituted (C₁-C₃)alkyl, benzyl, phenyl, hydroxy, or fluorine; or
R⁵ and R⁶, taken together with the nitrogen atom to which they are attached, form a 5- to 10-membered saturated heterocyclic radical containing at least one additional nitrogen atom, wherein
one or more of the carbon atoms of the heterocyclic radical is optionally substituted with (C₁-C₆)alkyl, (C₁-C₆)alkoxy, hydroxy, trifluoromethyl, or fluorine, and wherein
one or both of the additional nitrogen atoms of the heterocyclic radical is optionally substituted with (C₂-C₆)alkyl, and wherein
any carbon or nitrogen atom of the heterocyclic radical is optionally substituted with 2-pyridinyl, 3-pyridinyl, 4-pyridinyl, or a benzyl or phenyl group that is optionally substituted on the phenyl ring with one or more (C₁-C₆)alkyl, hydroxy, (C₁-C₆)alkoxy, trifluoromethyl, cyano, nitro, or halogen; or
R⁵ and R⁶, taken together with the nitrogen atom to which they are attached, form a 1-piperidinyl, 1-pyrrolidinyl, or 1-morpholino group, which is substituted with one or more (C₁-C₆)alkyl, (C₁-C₆)alkoxy, hydroxy, trifluoromethyl, fluorine, or a benzyl or phenyl group that is optionally substituted on the phenyl ring with one or more (C₁-C₆)alkyl, hydroxy, (C₁-C₆)alkoxy, trifluoromethyl, cyano, nitro, or halogen;
and pharmaceutical salts and esters thereof.

2. The compound of claim 1, wherein
R¹, R², R³, and R⁴ are defined as in claim 1;
R⁵ is hydrogen or (C₁-C₆)alkyl;
R⁶ is cyclohexyl which is substituted with one or more (C₁-C₃)alkyl, hydroxy, benzyloxy, (C₁-C₆)alkoxy, (C₁-C₆)alkyl-amino, bis[(C₁-C₃)alkyl]-amino, or fluorine,
(C₁-C₅)alkyl, optionally substituted with one or more cyclo(C₃-C₇)alkyl, hydroxy, benzyloxy, (C₁-C₆)alkoxy, (C₁-C₆)alkyl-amino, bis[(C₁-C₃)alkyl]-amino, or fluorine,
cyclopentyl, cycloheptyl or cyclo(C₃-C₇)alkyl-(C₁-C₃)alkyl, each of which may be optionally substituted with one or more (C₁-C₃)alkyl, hydroxy, benzyloxy, (C₁-C₆)alkoxy, (C₁-C₆)alkyl-amino, bis[(C₁-C₃)alkyl]-amino, or fluorine,
benzyl which is substituted on the phenyl ring with one or more fluorine, bromine, (C₁-C₆)alkyl, (C₁-C₆)alkoxy, trifluoromethyl, cyano, hydroxy, benzyloxy, or nitro,
phenyl substituted with one or more (C₁-C₆)alkyl, (C₁-C6)alkoxy, trifluoromethyl, cyano, hydroxy, benzyloxy, nitro, or halogen,
piperidin-4-yl, piperidin-3-yl, or pyrrolidin-3-yl, each of which may be optionally substituted on the nitrogen atom of the piperidine or pyrrolidine ring with (C₁-C₆)alkyl, hydroxy-substituted (C₁-C₆)alkyl, or a benzyl or phenyl group that is optionally substituted on the phenyl ring with one or more of (C₁-C₆)alkyl, (C₁-C₆)alkoxy, trifluoromethyl, cyano, hydroxy, or halogen,
—NR⁷R⁸
where R⁷ is hydrogen or (C₁-C₆)alkyl;
R⁸ is (C₁₋₂)alkyl, or a phenyl group that is optionally substituted with one or more (C₁-C₆)alkyl, (C₁-C₆)alkoxy, hydroxy-substituted (C₁-C₆)alkyl, hydroxy, trifluoromethyl, cyano, nitro, or halogen; or
R⁷ and R⁸, taken together with the nitrogen atom to which they are attached, form a 5- to 10-membered saturated heterocyclic radical which is optionally substituted by one or more (C₁-C₆)alkyl, (C₁-C₆)alkoxy, hydroxy-substituted (C₁-C₃)alkyl, benzyl, phenyl, hydroxy, or fluorine;
and pharmaceutical salts and esters thereof.

3. The compound of claim 2, wherein
R¹,R²,R³, and R⁴ are defined as in claim 1;
R⁵ is hydrogen;
R⁶ is cyclohexyl, trans-2-hydroxycyclohexyl, cis-2-hydroxycyclohexyl, 1-piperidinyl, 1-pyrrolidinyl, 1-azepanyl, 2-trifluoromethyl-phenylamino, or 4-trifluoromethyl-phenylamino;
and pharmaceutical salts and esters thereof.

4. The compound of claim 1, wherein
R¹, R², R³, and R⁴ are defined as in claim 1;
R⁵ and R⁶, taken together with the nitrogen atom to which they are attached, form a 5- to 10-membered saturated heterocyclic radical containing at least one additional nitrogen atom, wherein
one or more of the carbon atoms of the heterocyclic radical is optionally substituted with (C₁-C₆)alkyl, (C₁-C₆)alkoxy, hydroxy, trifluoromethyl, or fluorine, and wherein
one or both of the additional nitrogen atoms of the heterocyclic radical is optionally substituted with (C₂-C₆)alkyl, and wherein
any carbon or nitrogen atom of the heterocyclic radical is optionally substituted with 2-pyridinyl, 3-pyridinyl, 4-pyridinyl, or a benzyl or phenyl group that is optionally substituted on the phenyl ring with one or more (C₁-C₆)alkyl, hydroxy, (C₁-C₆)alkoxy, trifluoromethyl, cyano, nitro, or halogen;
and pharmaceutical salts and esters thereof.

5. The compound of claim 3, wherein
R¹, R², R³, and R⁴ are defined as in claim 1;
R⁵ and R⁶, taken together with the nitrogen atom to which they are attached, form a 1-piperazinyl group, wherein the nitrogen atom at the 4-position of the piperazine ring is optionally substituted with 2-pyridinyl, 3-pyridinyl, 4-pyridinyl, or a phenyl group that is optionally substituted on the phenyl ring with one or more (C₁-C₆)alkyl, (C₁-C₆)alkoxy, trifluoromethyl, cyano, or halogen;
and pharmaceutical salts and esters thereof.

6. The compound of claim 1, wherein
R¹, R², R³, and R⁴ are defined as in claim 1;
R⁵ and R⁶, taken together with the nitrogen atom to which they are attached, form a 1-piperidinyl, 1-pyrrolidinyl, or 1-morpholino group, which is substituted with one or more (C₁-C₆)alkyl, (C₁-C₆)alkoxy, hydroxy, trifluoromethyl, fluorine, or a benzyl or phenyl group that is optionally substituted on the phenyl ring with one or more (C₁-C₆)alkyl, hydroxy, (C₁-C₆)alkoxy, trifluoromethyl, cyano, nitro, or halogen;
and pharmaceutical salts and esters thereof.

7. The compound of claim 4, wherein
R¹, R², R³, and R⁴ are defined as in claim 1;
R⁵ and R⁶, taken together with the nitrogen atom to which they are attached, form a 1-piperidinyl group, which is substituted at the 4-position with a hydroxy and with a phenyl group that is optionally substituted on the phenyl ring with one or more (C₁-C₆)alkyl, (C₁-C₆)alkoxy, trifluoromethyl, cyano, or halogen;
and pharmaceutical salts and esters thereof.

8. A compound of Formula I 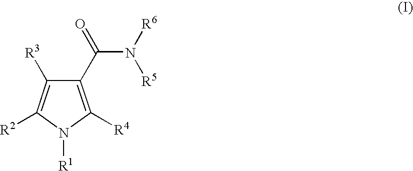
wherein
R¹ and R² are each a phenyl group optionally substituted with one or more halogen, (C₁-C₆)alkyl, (C₁-C₆)alkoxy, trifluoromethyl, cyano, or nitro;
R³ is hydrogen, (C₁-C₆)alkyl, or benzyl; and R⁴ is (C₂-C₆)alkyl or NH₂; or
R³ is (C₁-C₆)alkyl or benzyl; and R⁴ is CH₃;
R⁵ is hydrogen or (C₁-C₆)alkyl;
R⁶ is (C₁-C₉)alkyl, which is optionally substituted with one or more hydroxy, benzyloxy, (C₁-C₆)alkoxy, (C₁-C₆)alkyl-amino, bis[(C₁-C₃)alkyl]-amino, or fluorine,
benzyl, which is optionally substituted on the phenyl ring with one or more halogen, (C₁-C₆)alkyl, (C₁-C₆)alkoxy, trifluoromethyl, cyano, hydroxy, benzyloxy, or nitro,
phenyl substituted with one or more (C₁-C₆)alkyl, (C₁-C₆)alkoxy, trifluoromethyl, cyano, hydroxy, benzyloxy, nitro, or halogen,
piperidin-4-yl, piperidin-3-yl, or pyrrolidin-3-yl, each of which may be optionally substituted on the nitrogen atom of the piperidine or pyrrolidine ring with (C₁-C₆)alkyl, hydroxy-substituted (C₁-C₆)alkyl, or a benzyl or phenyl group that is optionally substituted on the phenyl ring with one or more (C₁-C₆)alkyl, (C₁-C₆)alkoxy, trifluoromethyl, cyano, hydroxy, or halogen,
—NR⁷R⁸
where R⁷ is hydrogen or (C₁-C₆)alkyl;
R⁸ is (C₁-C₆)alkyl, or a phenyl group that is optionally substituted with one or more (C₁-C₆)alkyl, (C₁-C₆)alkoxy, hydroxy-substituted (C₁-C₆)alkyl, hydroxy, trifluoromethyl, cyano, nitro, or halogen; or
R⁷ and R⁸, taken together with the nitrogen atom to which they are attached, form a 5- to 10-membered saturated heterocyclic radical which is optionally substituted by one or more (C₁-C₆)alkyl, (C₁-C₆)alkoxy, hydroxy-substituted (C₁-C₃)alkyl, benzyl, phenyl, hydroxy, or fluorine; or
R⁵ and R⁶, taken together with the nitrogen atom to which they are attached, form a 5- to 10-membered saturated heterocyclic radical, optionally substituted with one or more (C₁-C₆)alkyl, (C₁-C₆)alkoxy, hydroxy, trifluoromethyl, fluorine, or a benzyl or phenyl group that is optionally substituted on the phenyl ring with one or more (C₁-C₆)alkyl, hydroxy, (C₁-C₆)alkoxy, trifluoromethyl, cyano, nitro, or halogen;
and pharmaceutical salts and esters thereof.

9. The compound of claim 8, wherein
R¹, R⁷, R³, and R⁴ are defined as in claim 8;
R⁵ is hydrogen or (C₁-C₆)alkyl;
R⁶ is (C₁-C₉)alkyl, which is optionally substituted with one or more hydroxy, benzyloxy, (C₁-C₆)alkoxy, (C₁-C₆)alkyl-amino, bis[(C₁-C₃)alkyl]-amino, or fluorine,
benzyl, which is optionally substituted on the phenyl ring with one or more halogen, (C₁-C₆)alkyl, (C₁-C₆)alkoxy, trifluoromethyl, cyano, hydroxy, benzyloxy, or nitro,
phenyl substituted with one or more (C₁-C₆)alkyl, (C₁-C₆)alkoxy, trifluoromethyl, cyano, hydroxy, benzyloxy, nitro, or halogen,
piperidin-4-yl, piperidin-3-yl, or pyrrolidin-3-yl, each of which may be optionally substituted on the nitrogen atom of the piperidine or pyrrolidine ring with (C₁-C₆)alkyl, hydroxy-substituted (C₁-C₆)alkyl, or a benzyl or phenyl group that is optionally substituted on the phenyl ring with one or more (C₁-C₆)alkyl, (C₁-C₆)alkoxy, trifluoromethyl, cyano, hydroxy, or halogen,
—NR⁷R⁸
where R⁷ is hydrogen or (C₁-C₆)alkyl;
R⁸ is (C₁-C₉)alkyl, or a phenyl group that is optionally substituted with one or more (C₁-C₆)alkyl, (C₁-C₆)alkoxy, hydroxy-substituted (C₁-C₆)alkyl, hydroxy, trifluoromethyl, cyano, nitro, or halogen; or
R⁷ and R⁸, taken together with the nitrogen atom to which they are attached, form a 5- to 10-membered saturated heterocyclic radical which is optionally substituted by one or more (C₁-C₆)alkyl, (C₁-C₆)alkoxy, hydroxy-substituted (C₁-C₃)alkyl, benzyl, phenyl, hydroxy, or fluorine;
and pharmaceutical salts and esters thereof.

10. The compound of claim 9, wherein
R¹ and R² are defined as in claim 8;
R⁵ and R⁶ are defined as in claim 9;
R³ is hydrogen, (C₁-C₆)alkyl, or benzyl;
R⁴ is (C₂-C₆)alkyl or NH₂;
and pharmaceutical salts and esters thereof.

11. The compound of claim 9, wherein
R¹ and R² are defined as in claim 8;
R⁵ and R⁶ are defined as in claim 9;
R³ is (C₁-C₆)alkyl or benzyl;
R⁴ is CH₃;
and pharmaceutical salts and esters thereof.

12. The compound of claim 8, wherein
R¹,R², R³, and R are defined as in claim 8;
R⁵ and R⁶, taken together with the nitrogen atom to which they are attached, form a 5- to 10-membered saturated heterocyclic radical, optionally substituted with one or more (C₁-C₆)alkyl, (C₁-C₆)alkoxy, hydroxy, trifluoromethyl, fluorine, or a benzyl or phenyl group that is optionally substituted on the phenyl ring with one or more (C₁-C₆)alkyl, hydroxy, (C₁-C₆)alkoxy, trifluoromethyl, cyano, nitro, or halogen;
and pharmaceutical salts and esters thereof.

13. The compound of claim 12, wherein
R¹ and R² are defined as in claim 8;
R⁵ and R⁶ are defined as in claim 12;
R³ is hydrogen, (C₁-C₆)alkyl, or benzyl;
R⁴ is (C₂-C₆)alkyl or NH₂;
and pharmaceutical salts and esters thereof.

14. The compound of claim 12, wherein
R¹ and R² are defined as in claim 8;
R⁵ and R⁶ are defined as in claim 12;
R³ is (C₁₋₆)alkyl or benzyl;
R⁴ is CH₃;
and pharmaceutical salts and esters thereof.

15. The compound of claim 1 selected from the group consisting of:
2-amino-1-(2-chlorophenyl)-5-(4-chlorophenyl)-N-cyclohexyl-1H-pyrrole-3-carboxamide hydrochloride;
1-(2-chlorophenyl)-5-(4-chlorophenyl)-2-methyl-N 1-piperidinyl)-1H-pyrrole-3-carboxamide hydrochloride;
1-(2-chlorophenyl)-5-(4-methoxyphenyl)-2-methyl-M-[2-trifluoromethyl)phenyl]-1H-pyrrole-3-carbohydrazide hydrochloride;
1-(2-chlorophenyl)-5-(4-chlorophenyl)-2-methyl-N′-[2-trifluoromethyl)phenyl]-1H-pyrrole-3-carbohydrazide hydrochloride;
1-(2-chlorophenyl)-5-(4-methoxyphenyl)-2,4-dimethyl-N′-[4-(trifluoromethyl)phenyl]-1H-pyrrole-3-carbohydrazide hydrochloride;
5-(4-chlorophenyl)-I 2,4-dichlorophenyl)-N-[cis-2-hydroxycyclohexyl]-2-methyl-1H-pyrrole-3-carboxamide; and
1-(2-chlorophenyl)-54-chlorophenyl)-N-[trans-2-hydroxycyclohexyl]-2-methyl-1H-pyrrole-3-carboxamide.

16. A pharmaceutical composition comprising an effective amount of a compound of claim 1,
or a pharmaceutically acceptable salt or ester thereof, in combination with a pharmaceutically acceptable carrier.

17. A pharmaceutical composition comprising an effective amount of a compound of claim 15, or a pharmaceutically acceptable salt or ester thereof, in combination with a pharmaceutically acceptable carrier.

18. A pharmaceutical composition comprising an effective amount of a compound of claim 1,
or a pharmaceutically acceptable salt or ester thereof, in combination with a pharmaceutically acceptable carrier and one or more hypoglycemic agents.

19. The pharmaceutical composition of claim 18, wherein said hypoglycemic agent is selected from the group consisting of insulin, biguanidines, sulfonylureas, insulin secretagogues, α-glycosidase inhibitors, and β₃-adrenoreceptor agonists.

20. A pharmaceutical composition comprising an effective amount of a compound of claim 15, or a pharmaceutically acceptable salt or ester thereof, in combination with a pharmaceutically acceptable carrier and one or more hypoglycemic agents.

21. The pharmaceutical composition of claim 20, wherein said hypoglycemic agent is selected from the group consisting of insulin, biguanidines, sulfonylureas, insulin secretagogues, α-glycosidase inhibitors, and β₃-adrenoreceptor agonists.

22. A pharmaceutical composition comprising an effective amount of a compound of claim 1, or a pharmaceutically acceptable salt or ester thereof, in combination with a pharmaceutically acceptable carrier and one or more agents selected from the group consisting of HMG CoA reductase inhibitor, bile acid binding agent, fibric acid derivative, and agent that regulates hypertension.

23. A pharmaceutical composition comprising an effective amount of a compound of claim 15, or a pharmaceutically acceptable salt or ester thereof, in combination with a pharmaceutically acceptable carrier and one or more agents selected from the group consisting of HMG CoA reductase inhibitor, bile acid binding agent, fibric acid derivative, and agent that regulates hypertension.

24. A pharmaceutical composition comprising an effective amount of a compound of claim 1,
or a pharmaceutically acceptable salt or ester thereof, in combination with a pharmaceutically acceptable carrier and one or more agents selected from the group consisting of agents that modulate thermogenesis, lipolysis, gut motility, fat absorption, and satiety.

25. A pharmaceutical composition comprising an effective amount of a compound of claim 15, or a pharmaceutically acceptable salt or ester thereof, in combination with a pharmaceutically acceptable carrier and one or more agents selected from the group consisting of agents that modulate thermogenesis, lipolysis, gut motility, fat absorption, and satiety.

26. A composition comprising an effective amount of a compound of claim 1, or a salt or ester thereof, in combination with an inert carrier.

27. A composition comprising an effective amount of a compound of claim 15, or a salt or ester thereof, in combination with an inert carrier.

28. A method of treating obesity and obesity-related disorders comprising the step of administering to a patient in need thereof a pharmaceutically effective amount of a compound of claim 1.

29. The method of claim 28, wherein said obesity-related disorders include dyslipidemia, hypertriglyceridemia, hypertension, diabetes, Syndrome X, atherosclerotic disease, cardiovascular disease, cerebrovascular disease, peripheral vessel disease, cholesterol gallstones, cancer, menstrual abnormalities, infertility, polycystic ovaries, osteoarthritis, and sleep apnea.

30. A method of treating obesity and obesity-related disorders comprising the step of administering to a patient in need thereof a pharmaceutically effective amount of a compound of claim 15.

31. The method of claim 30, wherein said obesity-related disorders include dyslipidemia, hypertriglyceridemia, hypertension, diabetes, Syndrome X, atherosclerotic disease, cardiovascular disease, cerebrovascular disease, peripheral vessel disease, cholesterol gallstones, cancer, menstrual abnormalities, infertility, polycystic ovaries, osteoarthritis, and sleep apnea.

32. A method of regulating appetite and food intake comprising the step of administering to a patient in need thereof a pharmaceutically effective amount of a compound of claim 1.

33. A method of regulating appetite and food intake comprising the step of administering to a patient in need thereof a pharmaceutically effective amount of a compound of claim 15.

34. A method of treating bulimia comprising the step of administering to a patient in need thereof a pharmaceutically effective amount of a compound of claim 1.

35. A method of treating bulimia comprising the step of administering to a patient in need thereof a pharmaceutically effective amount of a compound of claim 15.

36. A method of treating obesity and obesity-related disorders comprising the step of administering to a patient in need thereof a pharmaceutically effective amount of a compound of claim 1 in combination with one or more hypoglycemic agents.

37. A method of treating obesity and obesity-related disorders comprising the step of administering to a patient in need thereof a pharmaceutically effective amount of a compound of claim 15 in combination with one or more hypoglycemic agents.

38. A method of treating obesity and obesity-related disorders comprising the step of administering to a patient in need thereof a pharmaceutically effective amount of a compound of claim 1 in combination with one or more agents that modulate digestion and/or metabolism.

39. The method of claim 38, wherein said agents that modulate digestion and/or metabolism include agents that modulate thermogenesis, lipolysis, gut motility, fat absorption, and satiety.

40. The method of claim 38, wherein said agents that modulate digestion and/or metabolism include β₃-adrenoreceptor agents.

41. A method of treating obesity and obesity-related disorders comprising the step of administering to a patient in need thereof a pharmaceutically effective amount of a compound of claim 15 in combination with one or more agents that modulate digestion and/or metabolism.

42. The method of claim 41, wherein said agents that modulate digestion and/or metabolism include agents that modulate thermogenesis, lipolysis, gut motility, fat absorption, and satiety.

43. The method of claim 41, wherein said agents that modulate digestion and/or metabolism include O₃-adrenoreceptor agents.

44. A method of treating obesity and obesity-related disorders comprising the step of administering to a patient in need thereof a pharmaceutically effective amount of a compound of claim 1 in combination with one or more agents selected from the group consisting of HMG CoA reductase inhibitor, bile acid binding agent, fibric acid derivative, and agent that regulates hypertension.

45. A method of treating obesity and obesity-related disorders comprising the step of administering to a patient in need thereof a pharmaceutically effective amount of a compound of claim 15 in combination with one or more agents selected from the group consisting of HMG CoA reductase inhibitor, bile acid binding agent, fibric acid derivative, and agent that regulates hypertension.

46. A method of treating CNS disorders comprising the step of administering to a patient in need thereof a pharmaceutically effective amount of a compound of claim 1.

47. A method of treating cognition and memory disorders comprising the step of administering to a patient in need thereof a pharmaceutically effective amount of a compound of claim 1.

48. A method of treating substance or behavioral addiction comprising the step of administering to a patient in need thereof a pharmaceutically effective amount of a compound of claim 1.

49. A method of treating CNS disorders comprising the step of administering to a patient in need thereof a pharmaceutically effective amount of a compound of claim 15.

50. A method of treating cognition and memory disorders comprising the step of administering to a patient in need thereof a pharmaceutically effective amount of a compound of claim 15.

51. A method of treating substance or behavioral addiction comprising the step of administering to a patient in need thereof a pharmaceutically effective amount of a compound of claim 15.
